(19) United States
(12) Patent Application Publication
(10) Pub. No.: US 2011/0319638 A1
(43) Pub. Date: Dec. 29, 2011

(54) ALUMINIUM COMPLEXES AND USE THEREOF AS A CATALYST IN INTRAMOLECULAR RING CLOSURE REACTIONS

(75) Inventors: Hisanori Itoh, Kanagawa (JP); Yoji Hori, Kanagawa (JP)

(73) Assignee: TAKASAGO INTERNATIONAL CORPORATION, Ohta-ku, Tokyo (JP)

(21) Appl. No.: 13/140,213

(22) PCT Filed: Dec. 17, 2009
(86) PCT No.: PCT/JP2009/071519

§ 371 Date: Jul. 11, 2011

(30) Foreign Application Priority Data
Dec. 17, 2008 (JP) 2008321114

(51) Int. Cl. C07C 29/56; C07F 5/06

(52) U.S. Cl. 549/210; 568/828

(57) ABSTRACT

To provide a process for increasing the proportion of an optical isomer of not only a compound having a closed ring but also a compound not having a closed ring when an optical isomer mixture of a compound having both a formyl group and a double bond capable of causing a carbonyl-ene ring closing reaction in the same molecule is subjected to a ring closing reaction. A process for increasing the proportion of an optical isomer characterized by subjecting an optical isomer mixture of a compound having both a formyl group and a double bond capable of causing a carbonyl-ene ring closing reaction in the same molecule to a ring closing reaction in the presence of a predetermined aluminum complex represented by the general formula: [Al₁(L¹)₁(L²)ₘ(Lh)ₙ]ₖ.

### TECHNICAL FIELD

The present invention relates to a process for producing isopulegol and an analogous compound thereof, which are useful as a raw material for a flavor or fragrance etc. and an important precursor for synthesizing menthol. By subjecting an optical isomer mixture of a compound having both a formyl group and a double bond capable of causing a carbonyl-ene ring closing reaction in the same molecule to a ring closing reaction using a novel asymmetric aluminum complex as a catalyst, the present invention can increase the proportion of either d-form or l-form of a compound produced by the ring closing reaction, or the proportion of either d-form or l-form of the optical isomer mixture which is not reacted by ring closure.

In particular, when only one of the optical isomers of citronellal having low enantiomeric excess is preferentially reacted, thereby increasing the proportion of the stereoisomer, optical resolution of citronellal is successfully made. Alternatively, when a substrate-selective ring closing reaction is conducted, isopulegol having an increased proportion of a predetermined optical isomer which is not reacted by ring closure can be obtained.

### BACKGROUND ART

Menthol, particularly, l-menthol, is conventionally a very important flavor or fragrance having a pleasant cooling sensation and is applied to a wide variety of uses. As a process for obtaining l-menthol, a process of optically resolving dl-menthol and an asymmetric synthesis process are known (Synthetic flavor, written by Motoichi Indo, The Chemical Daily Co., Ltd, pp. 106 to 114). In a production step for l-menthol by the asymmetric synthesis, l-isopulegol as a precursor is hydrogenated to obtain l-menthol. In a step of synthesizing the l-isopulegol, a selective ring closing reaction of d-citronellal is important.

As to the selective ring closing reaction of d-citronellal, various processes have long been known. As a highly selective reaction using an aluminum complex as a catalyst, recently, a highly selective ring closing reaction using an aluminum complex having a ligand derived from 2,6-diphenylphenoxy moiety (Japanese Patent Application Laid-Open No. 2002-212121) has been found. Other than this, a ring closing reaction (WO2006/069659, WO2006/092433, DE102005023953) using an aluminum complex having a ligand derived from a compound having a phenoxy moiety as a catalyst, and a ring closing reaction (WO2007/039342) using an aluminum complex having a siloxy moiety as a catalyst have been reported. However, there is no report on a reaction for selectively closing a ring of one of the optical isomers from racemic citronellal by using an optically active aluminum complex. Furthermore, there are many reports on an aluminum catalyst having a diol skeleton serving as an asymmetric ligand and derived from tartaric acid (U.S. Pat. No. 6,166,260, Synlett, 1998, 1291-1293, Tetrahedron: Asymmetry 1991, Vol. 2, No. 12, 1295-1304, CROATIA CHEMICA ACTA, 1996, 69, 459-484, Russian Chemical Bulletin, 2000, 49, 460-465); however, each of them relates to only a cationic complex or an aluminum complex having a specific substituent such as a halogen group or an aminohydroxy group. There are no report on a neutral complex having a ratio of aluminum to diol (which is an asymmetric ligand derived from tartaric acid) of 1:1 and having an alkyl group or an alkoxy group or an aluminum neutral complex having a ratio of aluminum to diol of 2:3.

### SUMMARY OF INVENTION

An object of the present invention is to provide a process for obtaining a desired optically active alcohol or optically active olefin aldehyde enhanced in optical purity by causing an intramolecular carbonyl-ene ring closing reaction using a novel asymmetric aluminum complex as a catalyst, thereby increasing the proportion of a predetermined optical isomer of a compound produced by the ring closure or a compound left unreacted, more specifically, to provide a process for obtaining l-isopulegol and l-citronellal or d-isopulegol and d-citronellal by optical resolution of citronellal by a highly selective ring closing reaction.

The present inventors have conducted intensive studies with a view to attaining the above objects. As a result, they found that when a specific catalyst is used, citronellal corresponding to the configuration of an asymmetric ligand can be preferentially ring-closed, with the result that a dl enantio selectivity is improved and further isopulegol is highly selectively (an isomer ratio of 80% or more) obtained from four types of isomers, namely, isopulegol, isoisopulegol, neoisopulegol and neoisoisopulegol, in a high yield. They continued further investigation and accomplished the present invention.

To be more specific, the present invention encompasses the following inventions.

[1] An aluminum complex represented by the general formula (1′) below:

[Alₗ(L¹)ₗ(L²)ₘ(Lh)ₙ]ₖ (1′)

wherein in the formula (1′), l represents an integer of 1 or 2, with the proviso that when l=1, m=0 and n=1, and when l=2, m=1 and n=0; k represents a natural number; L¹ represents a ligand represented by the formula (2-A′) below or the formula (2-B′) below; L² represents a ligand represented by the formula (3-A′) or (3-B′) below; Lh represents an alkyl group, an alkoxy group, a carboxy group, a siloxy group, an amino group, a fluorine atom, a bromine atom or an iodine atom,

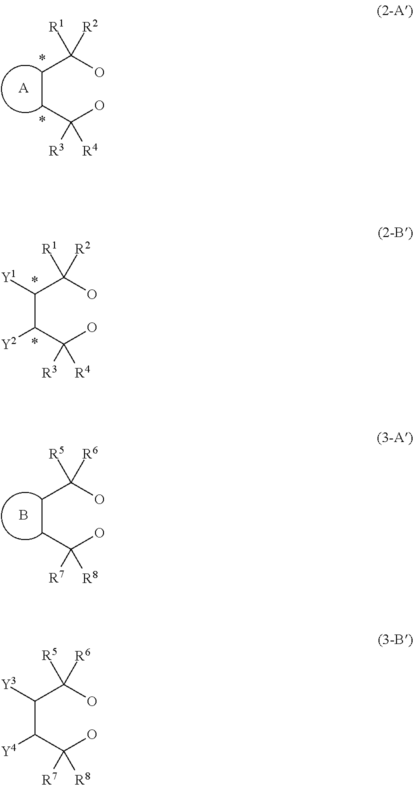

in the formula (2-A′), R¹, R², R³ and R⁴ each independently represent an aryl group that may have a substituent, a heterocyclic group that may have a substituent, an aliphatic chain that may have a substituent or an alicyclic group that may have a substituent, and R¹ and R², and R³ and R⁴ may be taken together to form a ring, and ring A is a 3- to 8-membered ring that may have a hetero element and symbol * represents an optically active asymmetric carbon atom,

in the formula (2-B′), R¹, R², R³ and R⁴ each independently represent an aryl group that may have a substituent, a heterocyclic group that may have a substituent, an aliphatic chain that may have a substituent or an alicyclic group that may have a substituent. R¹ and R², and R³ and R⁴ may be taken together to form a ring. Y¹ and Y² each independently represent an aliphatic chain that may have a substituent, an alicyclic group that may have a substituent, an aryl group that may have a substituent, a heterocyclic group that may have a substituent, an alkoxy group, a siloxy group that may have a substituent or a carboxy group, and symbol * represents an optically active asymmetric carbon atom,

in the formula (3-A′), R⁵, R⁶, R⁷, and R⁸ each independently represent an aryl group that may have a substituent, a heterocyclic group that may have a substituent, an aliphatic chain that may have a substituent or an alicyclic group that may have a substituent, and R⁵ and R⁶, and R⁷ and R⁸ may be taken together to form a ring, and ring B is a 3- to 8-membered ring that may have a hetero element, and

in the formula (3-B′), R⁵, R⁶, R⁷ and R⁸ each independently represent an aryl group that may have a substituent, a heterocyclic group that may have a substituent, an aliphatic chain that may have a substituent, or an alicyclic group that may have a substituent. R⁵ and R⁶, and R⁷ and R⁸ may be taken together to form a ring. Y³ and Y⁴ each independently represent an aliphatic chain that may have a substituent, an alicyclic group that may have a substituent, an aryl group that may have a substituent, a heterocyclic group that may have a substituent, an alkoxy group, a siloxy group that may have a substituent, or a carboxy group.

[2] A process for producing the aluminum complex according to item [1], said process comprising the step of reacting an aluminum compound represented by the general formula (1) below:

Al(Lg)₃ (1)

wherein in the formula (1), Lg represents an alkyl group, an alkoxy group or a halogen atom,

with a diol compound represented by the general formula (2-A) below or the general formula (2-B) below:

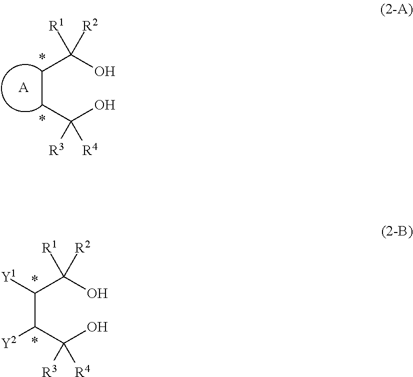

wherein in the formula (2-A), R¹, R², R³, R⁴, ring A and symbol * have the same meanings as defined in the formula (2-A′) of item [1], and in the formula (2-B), R¹, R², R³, R⁴, Y¹, Y² and symbol * have the same meanings as defined in the formula (2-B′) of item [1].

[3] The process for producing the aluminum complex according to item [2], wherein in the reaction an additive is added.

[4] A process for producing the aluminum complex according to item [1], said process comprising the step of reacting an aluminum compound represented by the general formula (1) below:

Al(Lg)₃ (1)

wherein in the formula (1), Lg represents an alkyl group, an alkoxy group or a halogen atom,

with a diol compound represented by the general formula (2-A) below or the general formula (2-B) below, and a diol compound represented by the general formula (3-A) below or the general formula (3-B) below:

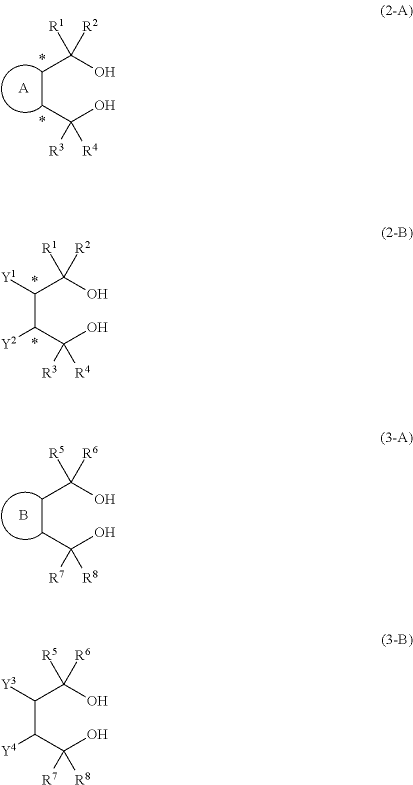

wherein in the formula (2-A), R¹, R², R³, R⁴ ring A and symbol * have the same meanings as defined in the formula (2-A′) of item [1],

in the formula (2-B), R¹, R², R³, R⁴, Y¹, Y² and symbol * have the same meanings as defined in the formula (2-B′) of item [1],

in the formula (3-A), R⁵, R⁶, R⁷, R⁸ and ring B have the same meanings as defined in the formula (3-A′) of item [1], and

in the formula (3-B), R⁵, R⁶, R⁷, R⁸, Y³ and Y⁴ have the same meanings as defined in the formula (3-B′) of item [1].

[5] The process for producing the aluminum complex according to items [2] to [4], wherein the diol compound represented by the general formula (2-A) or the general formula (2-B) is an optically active substance derived from tartaric acid.

[6] The process for producing the aluminum complex according to items [4] and [5], in which the diol compound represented by the general formula (3-A) or the general formula (3-B) is an optically active substance derived from tartaric acid.

[7] A process for producing an optically active compound, said process comprising the step of subjecting an optical isomer mixture of a compound having both a formyl group and a double bond capable of causing a carbonyl-ene ring closing reaction in the same molecule to a ring closing reaction in the presence of the aluminum complex according to item [1], wherein the optically active compound is enriched with either a d-form or l-form compound produced by the ring closing reaction of the compound having both the formyl group and the double bond.

[8] The production process according to item [7], in which the compound having both the formyl group and the double bond capable of causing the carbonyl-ene ring closing reaction in the same molecule is a compound represented by the general formula (4) below:

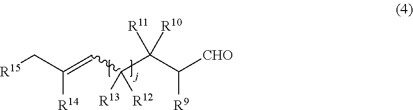

wherein in the formula (4), j represents an integer of 1 or 2; R⁹, R¹⁰, and R¹² each independently represent a hydrogen atom or an alkyl group that may have a substituent; R¹¹ represents an alkyl group that may have a substituent or a hydroxy group that may be protected with a protecting group; R¹³, R¹⁴ and R¹⁵ each independently represent a hydrogen atom or an alkyl group that may have a substituent; and the wavy line represents an E or Z conformation.

[9] The production process according to item [7], in which the compound produced by ring closure is a compound represented by the general formula (5) below:

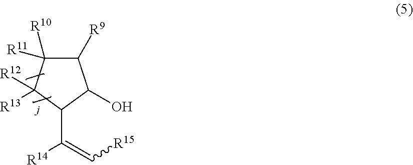

wherein in the formula (5), j represents an integer of 1 or 2; R⁹, R¹⁰ and R¹² each independently represent a hydrogen atom or an alkyl group that may have a substituent; R¹¹ represents an alkyl group that may have a substituent or a hydroxy group that may be protected with a protecting group; R¹³, R¹⁴ and R¹⁵ each independently represent a hydrogen atom or an alkyl group that may have a substituent; and the wavy line represents an E or Z conformation.

[10] The production process according to item [7], in which the compound having both the formyl group and the double bond capable of causing the carbonyl-ene ring closing reaction in the same molecule is optically active citronellal and the compound produced by ring closure is optically active isopulegol.

[11] The production process according to item [10], in which the optically active isopulegol is l-isopulegol.

[12] The production process according to item [10], in which the optically active citronellal is l-citronellal.

[13] A process for enriching either d-form or l-form in an optical isomer mixture of a compound having both a formyl group and a double bond capable of causing a carbonyl-ene ring closing reaction in the same molecule, said process comprising the step of subjecting the optical isomer mixture to a ring closing reaction in the presence of the aluminum complex according to item [1], wherein either d-form or l-form is not reacted by ring closure.

According to the present invention, it is possible to obtain a desired optically active alcohol or optically active olefin aldehyde enhanced in optical purity by conducting an intramolecular carbonyl-ene ring closing reaction using a novel aluminum complex as a catalyst, thereby increasing the proportion of a predetermined optical isomer of a compound produced by the ring closure or a compound left unreacted.

### BRIEF DESCRIPTION OF THE DRAWINGS

FIG. 1 shows an NMR chart of a solid substance obtained in Example 1;

FIG. 2 shows an enlarged chart of a low magnetic field side of the NMR chart shown in FIG. 1;

FIG. 3 shows an NMR chart of (R,R)-TADDOL;

FIG. 4 shows an enlarged chart of a low magnetic field side of the NMR chart shown in FIG. 3;

FIG. 5 shows an NMR chart of a solid substance obtained in Example 2;

FIG. 6 shows an enlarged chart of a low magnetic field side of the NMR chart shown in FIG. 5;

FIG. 7 shows an NMR chart of (R,R)-1-naphthyl TADDOL; and

FIG. 8 shows an enlarged chart of a low magnetic field side of the NMR chart shown in FIG. 7.

### DESCRIPTION OF EMBODIMENTS

The present invention will be more specifically described below.

In an aluminum compound represented by general formula (1) to be used for preparing the aluminum catalyst of the present invention, Lg represents an alkyl group, an alkoxy group or a halogen atom.

The alkyl group represented by Lg includes a linear or branched alkyl group having 1 to 8 carbon atoms, preferably 1 to 4 carbon atoms. Specific examples thereof include a methyl group, an ethyl group, a propyl group, an isopropyl group, a n-butyl group, a sec-butyl group, an isobutyl group and a tert-butyl group.

The alkoxy group represented by Lg includes besides an aliphatic alkoxy group, an aryloxy group, an aralkyloxy group and the like. The aliphatic alkoxy group includes a linear or branched alkoxy group having 1 to 8 carbon atoms, preferably 1 to 4 carbon atoms. Specific examples thereof include a methoxy group, an ethoxy group, a propoxy group, an isopropoxy group, a n-butoxy group, a sec-butoxy group, an isobutoxy group, a tert-butoxy group and the like. The aryloxy group includes an aryloxy group having 6 to 14 carbon atoms, preferably 6 to 10 carbon atoms. Specific examples thereof include a phenoxy group, a naphthoxy group and the like. The aralkyloxy group includes an aralkyloxy group having 7 to 15 carbon atoms, preferably 7 to 11 carbon atoms. Specific examples thereof include a benzyloxy group, a 1-phenethyloxy group and the like.

The halogen atom represented by Lg includes a fluorine atom, a chlorine atom, a bromine atom, an iodine atom and the like.

The groups represented by Lg may be the same or different, and two of three groups may be the same.

Lg is not necessarily an optically active substance.

Preferable examples of an aluminum compound represented by the general formula (1) include trimethylaluminum, triethylaluminum, triisopropylaluminum, tri-n-propylaluminum, tri-n-butylaluminum, triisobutylaluminum, tri-sec-butylaluminum, tri-t-butylaluminum, trimethoxyaluminum, triethoxyaluminum, triisopropoxyaluminum, tri-n-propoxyaluminum, tri-n-butoxyaluminum, tri-sec-butoxyaluminum, tri-t-butoxyaluminum, aluminum trichloride, aluminum tribromide, aluminum triiodide, aluminum trifluoride and the like.

In diol compounds represented by the general formulas (2-A) and (2-B), and ligands derived from the diol compounds and represented by the general formulas (2-A′) and (2-B′), R¹, R², R³ and R⁴ each independently represent an aryl group that may have a substituent, a heterocyclic group that may have a substituent, an aliphatic chain that may have a substituent or an alicyclic group that may have a substituent. R¹ and R² or R³ and R⁴ may be taken together to form a ring. In a diol compound represented by the general formula (2-B) and a ligand derived from the diol compound and represented by the general formula (2-B′), Y¹ and Y² each independently represent an aliphatic chain that may have a substituent, an alicyclic group that may have a substituent, an aryl group that may have a substituent, a heterocyclic group that may have a substituent, an alkoxy group, a siloxy group that may have a substituent or a carboxy group. In a diol compound represented by the general formula (2-A) and a ligand derived from the diol compound and represented by the general formula (2-A′), ring A is a 3- to 8-membered ring that may have a hetero element.

In diol compounds represented by the general formulas (3-A) and (3-B) and the ligands derived from the diol compounds and represented by the general formulas (3-A′) and (3-B′), R⁵, R⁶, R⁷ and R⁸ each independently represent an aryl group that may have a substituent, a heterocyclic group that may have a substituent, an aliphatic chain that may have a substituent or an alicyclic group that may have a substituent. R⁵ and R⁶ or R⁷ and R⁸ may be taken together to form a ring. In a diol compound represented by the general formula (3-B) and a ligand derived from the diol compound and represented by the general formula (3-B′), Y³ and Y⁴ each independently represent an aliphatic chain that may have a substituent, an alicyclic group that may have a substituent, an aryl group that may have a substituent, a heterocyclic group that may have a substituent, an alkoxy group, a siloxy group that may have a substituent or carboxy group. In a diol compound represented by the general formula (3-A) and a ligand derived from the diol compound and represented by the general formula (3-A′), ring B is a 3- to 8-membered ring that may have a hetero element.

In diol compounds represented by the general formulas (2-A), (2-B), (3-A) and (3-B) and ligands derived from the diol compounds and represented by the general formulas (2-A′), (2-B′), (3-A′) and (3-B′), groups represented by R¹, R², R³, R⁴, R⁵, R⁶, R⁷ and R⁸ will be described.

The aryl group that may have a substituent includes an aryl group having 6 to 14 carbon atoms, preferably 6 to 10 carbon atoms. Specific examples thereof include a phenyl group, a naphthyl group, an anthranyl group, a phenanthryl group and the like. The substituent that the aryl group has includes an alkyl group having 1 to 4 carbon atoms, an alkoxy group having 1 to 4 carbon atoms and the like, as well as polymer chains such as a 6,6-nylon chain, a vinyl polymer chain, and a styrene polymer chain.

A heterocyclic group that may have a substituent includes an aliphatic heterocyclic group having 2 to 14 carbon atoms such as a piperidino group, a piperazinyl group, a morpholino group, a tetrahydrofuryl group, a tetrahydropyranyl group and a tetrahydrothienyl group; an aromatic heterocyclic group having 4 to 14 carbon atoms such as a furyl group, a thienyl group, a pyridyl group, a pyrimidyl group, a pyrazyl group, a pyridazyl group, a pyrazolyl group, an imidazolyl group, an oxazolyl group, a thiazolyl group, a benzofuryl group, a benzothienyl group, a quinolyl group, an isoquinolyl group, a quinoxalyl group, a phthalazyl group, a quinazolyl group, a naphthyridyl group, a chinolyl group, a benzoimidazolyl group, a benzooxazolyl group and a benzothiazolyl group; and the like. The substituent that the heterocyclic group has includes an alkyl group having 1 to 4 carbon atoms, an alkoxy group having 1 to 4 carbon atoms and the like, as well as polymer chains such as a 6,6-nylon chain, a vinyl polymer chain, and a styrene polymer chain.

The aliphatic chain that may have a substituent includes a linear or branched alkyl group having 1 to 8 carbon atoms, preferably 1 to 4 carbon atoms. Specific examples thereof include a methyl group, an ethyl group, a propyl group, an isopropyl group, a n-butyl group, a sec-butyl group, an isobutyl group and a tert-butyl group. The substituent that the aliphatic chain has includes an alkyl group having 1 to 4 carbon atoms, an alkoxy group having 1 to 4 carbon atoms and the like, as well as polymer chains such as a 6,6-nylon chain, a vinyl polymer chain, and a styrene polymer chain.

The alicyclic group that may have a substituent includes an alicyclic group having 3 to 14 carbon atoms, preferably 3 to 8 carbon atoms. Specific examples thereof include a cyclopropyl group, a cyclobutyl group, a cyclopentyl group, a cyclohexyl group, a cycloheptyl group, a cyclooctyl group and the like. The substituent that the alicyclic group has includes an alkyl group having 1 to 4 carbon atoms, an alkoxy group having 1 to 4 carbon atoms and the like, as well as polymer chains such as a 6,6-nylon chain, a vinyl polymer chain, and a styrene polymer chain.

In diol compounds represented by the general formulas (2-B) and (3-B) and the ligands derived from the diol compounds and represented by the general formulas (2-B′) and (3-B′), groups represented by Y¹, Y², Y³ and Y⁴ will be described.

The aliphatic chain that may have a substituent, the alicyclic group that may have a substituent, the aryl group that may have a substituent and the heterocyclic group that may have a substituent includes the same examples as those mentioned for R¹, R², R³, R⁴, R⁵, R⁶, R⁷ and R⁸ above.

The alkoxy group includes besides an aliphatic alkoxy group, an aryloxy group, an aralkyloxy group and the like. The aliphatic alkoxy group includes a linear or branched alkoxy group having 1 to 8 carbon atoms, which may have a ring structure. Specific examples thereof include a methoxy group, an ethoxy group, a propoxy group, an isopropoxy group, a n-butoxy group, a sec-butoxy group, an isobutoxy group, a tert-butoxy group, a cyclohexyl group and a n-octyl group. The aryloxy group includes an aryloxy group having 6 to 14 carbon atoms, preferably 6 to 10 carbon atoms. Specific examples thereof include a phenoxy group, a naphthoxy group and the like. The aralkyloxy group includes an aralkyloxy group having 7 to 15 carbon atoms, preferably 7 to 11 carbon atoms. Specific examples thereof include a benzyloxy group, a 1-phenethyloxy group and the like.

The siloxy group that may have a substituent includes a siloxy group having a hydrocarbon substituent having 1 to 12 carbon atoms. Specific examples thereof include a trimethylsiloxy group, a triethylsiloxy group, a triisopropylsiloxy group, a triphenylsiloxy group, a dimethyl-tert-butylsiloxy group, a diethylphenylsiloxy group and a diphenyl-tert-butylsiloxy group. The substituent that the siloxy group has includes an alkyl group having 1 to 4 carbon atoms, an alkoxy group having 1 to 4 carbon atoms, a silyl group, a siloxy group and the like, as well as a polymer chains such as a 6,6-nylon chain, a vinyl polymer chain, and a styrene polymer chain.

The carboxy group includes a carboxy group derived from a carboxylic acid, for example, a carboxy group having 2 to 18 carbon atoms. Specific examples thereof include an acetoxy group, a propionyloxy group, an acryloyloxy group, a butyryloxy group, a pivaloyloxy group, a pentanoyloxy group, a hexanoyloxy group, a lauroyloxy group, a stearoyloxy group and a benzoyloxy group.

Ring A in the general formulas (2-A) and the general formula (2-A′) and ring B in the general formulas (3-A) and the general formula (3-A′) will be described.

Ring A and ring B are 3- to 8-membered rings that may have a hetero element.

The hetero element in ring A and ring B includes sulfur, oxygen, nitrogen, boron, silicon, other metal elements capable of forming a metallacycle and the like. A plurality of hetero elements may be present in ring A and ring B. In this case, the hetero elements may be the same or different.

Ring A and ring B may have a substituent and the hetero element may have a substituent.

Specific examples of the ring A and ring B include a benzene ring, a cyclopropane ring, a cyclobutane ring, a cyclopentane ring, a cyclohexane ring, a cycloheptane ring, a cyclooctane ring, a cyclohexene ring, a norbornane ring, a norbornene ring, a tetrahydrofuran ring, a dioxolane ring, a dioxane ring, a dioxacycloheptane ring, a trioxacycloheptane ring, a lactone ring, a lactam ring, a morpholine ring, a pyrrolidine ring, a piperidine ring, a tetrahydrothiophene ring and the like.

The substituents that these ring structures can have include an alkyl group, an aryl group, an aralkyl group, an alkoxy group, a vinyl polymer chain, a styrene polymer chain and the like.

The diol compounds represented by the general formula (2-A) and the general formula (3-A) and ligands represented by the general formula (2-A′) and the general formula (3-A′) may form a polymer chain via a substituent and a carbon chain that ring A and ring B have.

Preferable specific examples of the diol compounds represented by the general formula (2-A) and the general formula (3-A) of the present invention include, but are not limited to, the following compounds.

The diol compound represented by the general formula (2-A) is an optically active substance.

The diol compound represented by the general formula (3-A) may be an optically active substance or a racemic mixture.

In the following compounds, Et represents an ethyl group. The same shall apply hereinafter.

In the following compounds, symbol * represents a polymer chain bond. Symbol ∘ indicates 1 to 500.

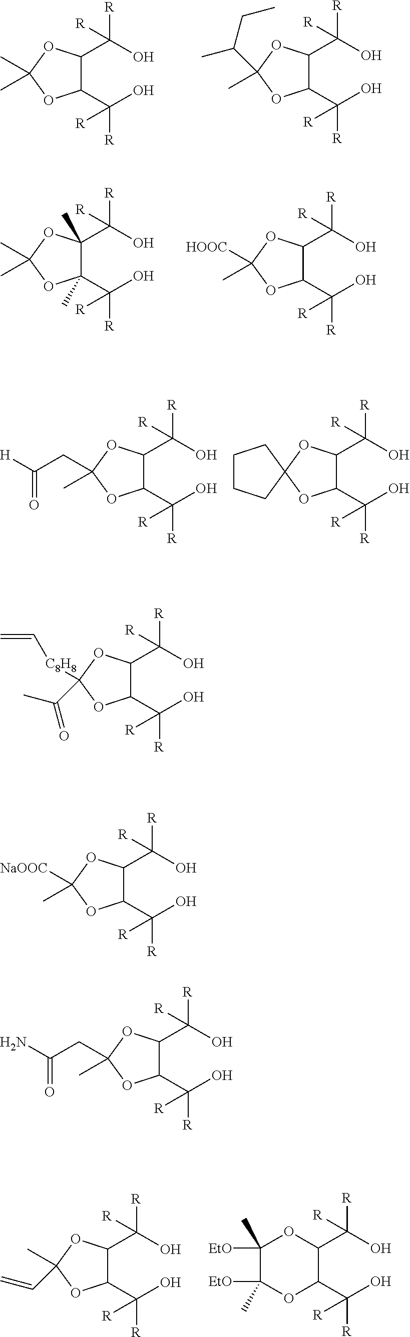

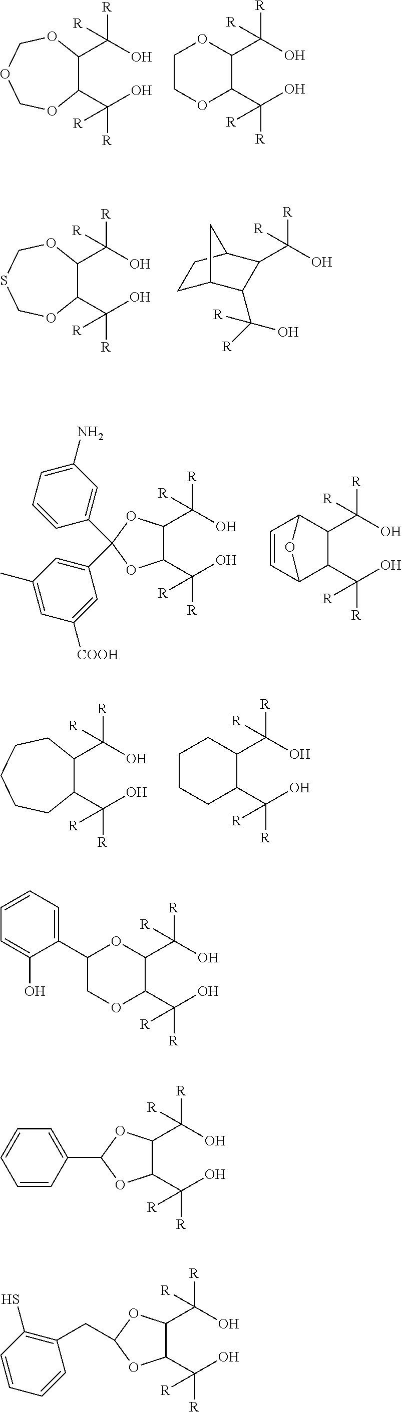

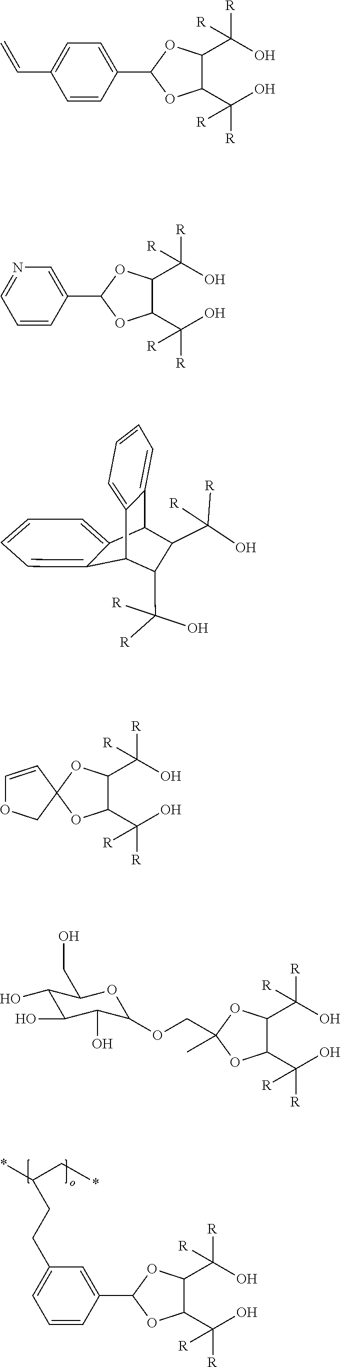

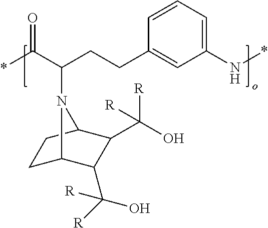

In the aforementioned compounds, R represents a substituent. Specific examples of R are shown below. The four substituents represented by R in the above compounds may be the same or different. Two or three of the four substituents may be the same.

In the substituents below, Me represents a methyl group, and Ph represents a phenyl group. The same shall apply hereinafter.

In the substituents below, symbol * represents a binding site, and symbol ** represents a polymer chain bond. Symbol ∘ indicates 1 to 500.

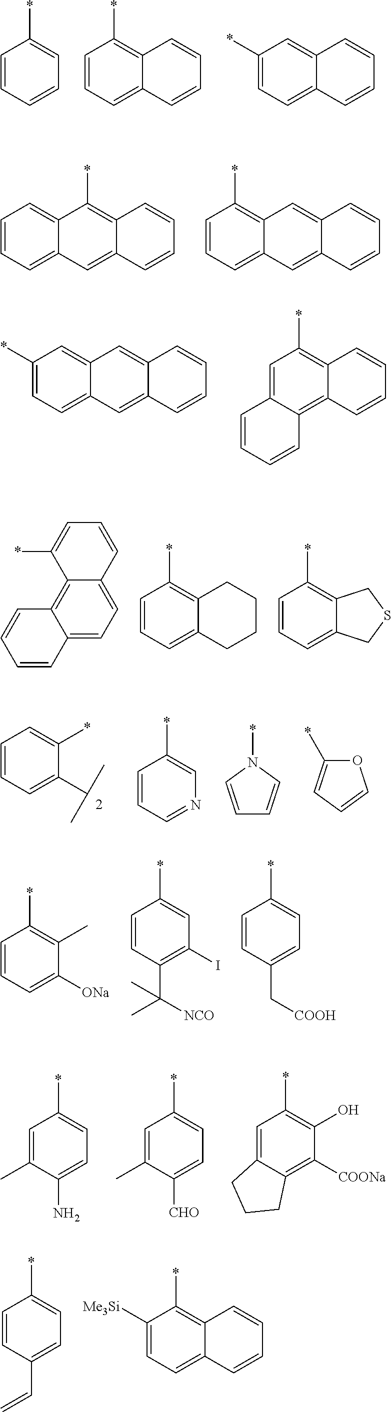

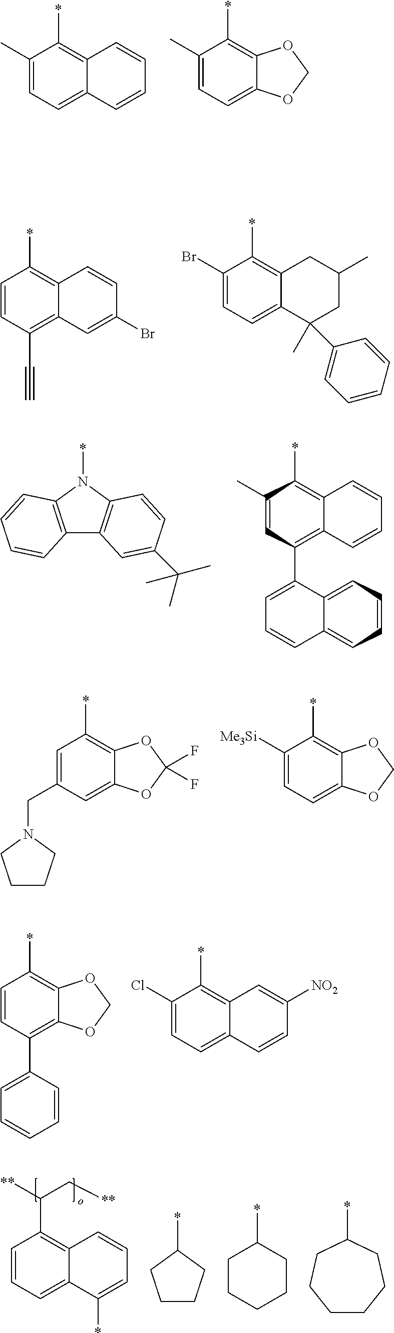

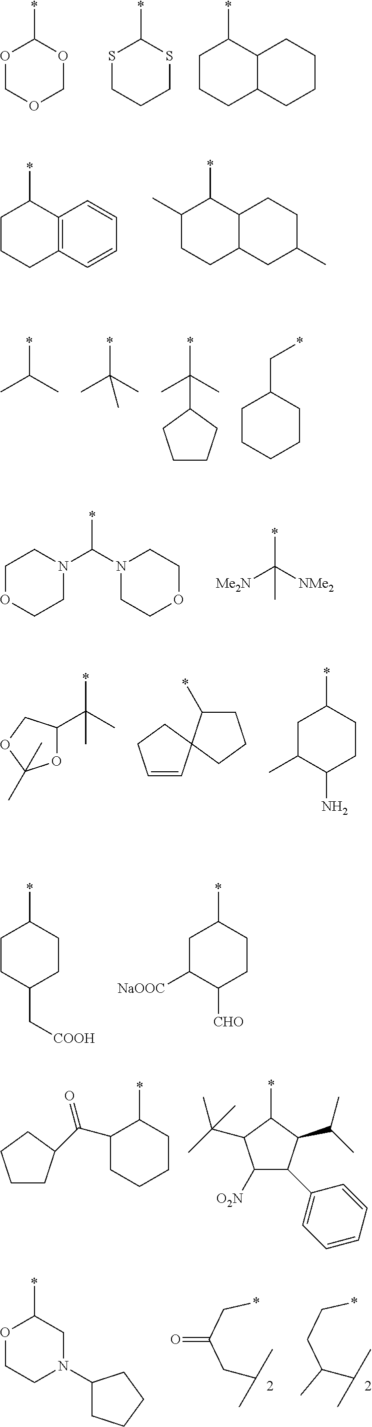

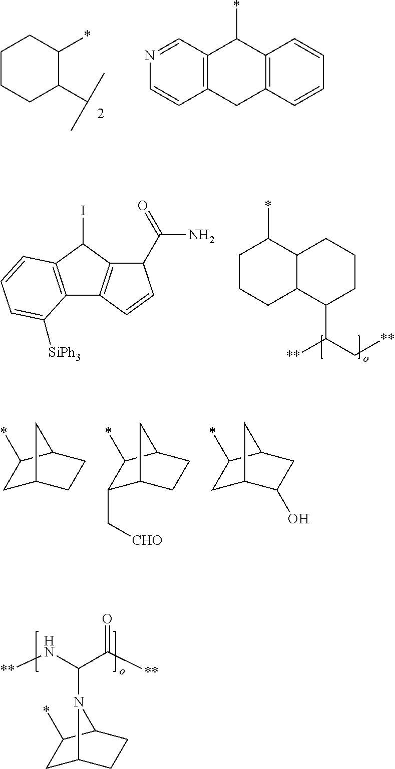

Specific examples of the ligands represented by the general formula (2-A′) and the general formula (3-A′) include, but are not limited to, compounds derived from the specific examples of the diol compound represented by the general formula (2-A) and the general formula (3-A) above.

The ligand represented by the general formula (2-A′) is an optically active substance.

The ligand represented by the general formula (3-A′) may be an optically active substance or a racemic mixture.

Specific examples of a diol compound represented by the general formula (2-B) and the general formula (3-B) preferably include, but are not limited to, the following compounds.

In the following compounds, R represents a substituent. Specific examples of the substituent R are the same as the aforementioned specific examples of R. The four substituents represented by R in the following compounds may be the same or different. Two or three of the four substituents may be the same.

The diol compound represented by the general formula (2-B) is an optically active substance.

The diol compound represented by the general formula (3-B) may be an optically active substance or a racemic mixture.

In the following compounds, symbol * represents a polymer chain bond.

Symbol ∘ indicates 1 to 500.

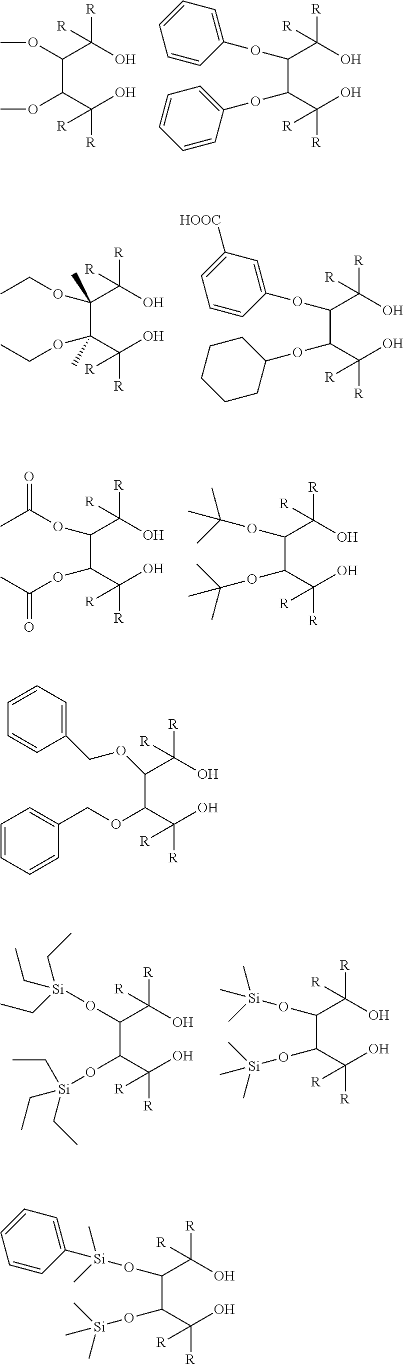

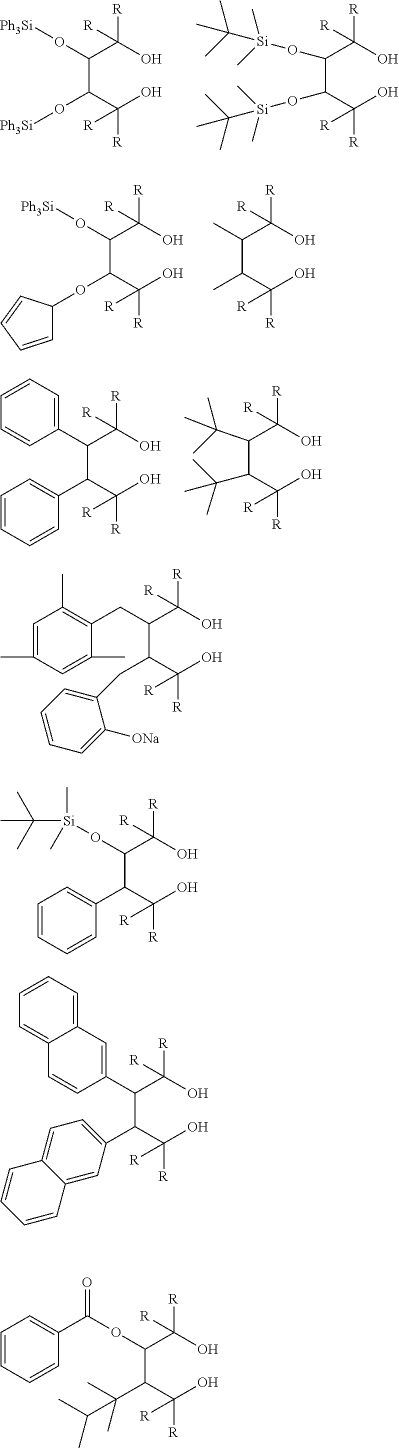

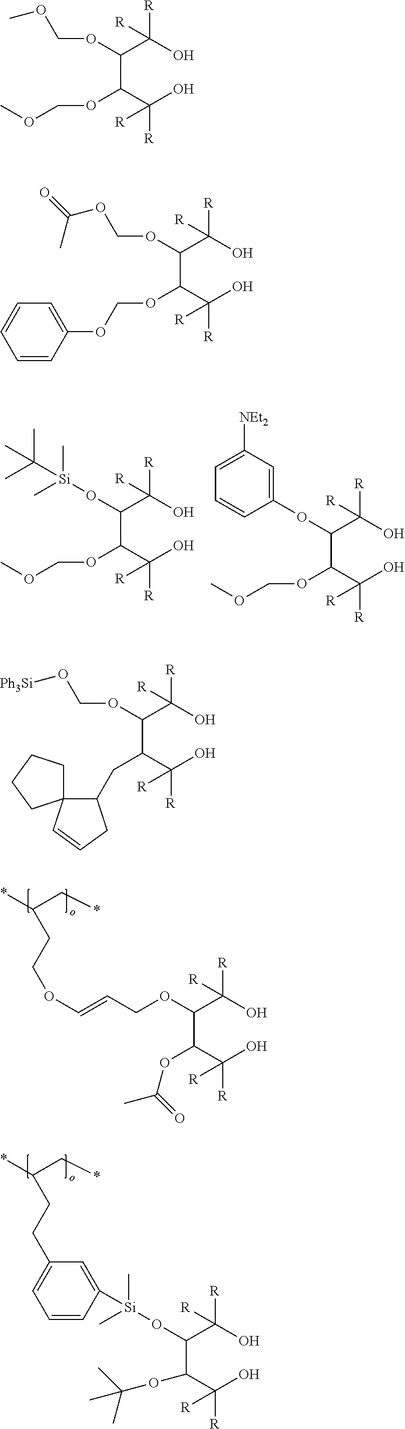

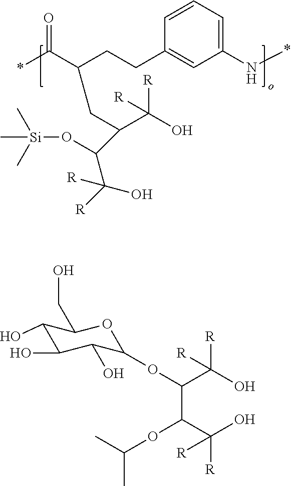

Specific examples of the ligands represented by the general formula (2-B′) and the general formula (3-B′) include, but are not limited to, ligands derived from the aforementioned specific examples of the compound represented by the general formula (2-B) and the general formula (3-B).

The ligand represented by the general formula (2-B′) is an optically active substance.

The ligand represented by the general formula (3-B′) may be an optically active substance or a racemic mixture.

An aluminum complex represented by the general formula (1′) will be described.

[Alₗ(L¹)ₖ(L²)ₘ(Lh)ₙ]ₖ (1′)

In the general formula (1′), l represents an integer of 1 or 2, with the proviso that when l=1, m=0 and n=1, and when l=2, m=1 and n=0; k represents a natural number, preferably 1 to 10; L¹ represents a ligand represented by the general formula (2-A′) or the general formula (2-B′); L² represents a ligand represented by the general formula (3-A′) or the general formula (3-B′).

In an aluminum complex represented by the general formula (1′), Lh represents an alkyl group, an alkoxy group, a carboxy group, a siloxy group, an amino group, a fluorine atom, a bromine atom or an iodine atom.

The alkyl group includes a linear, branched or cyclic alkyl group having 1 to 8 carbon atoms, preferably 1 to 4 carbon atoms. Specific examples thereof include a methyl group, an ethyl group, a propyl group, an isopropyl group, a n-butyl group, a sec-butyl group, an isobutyl group, a tert-butyl group, a cyclopropyl group, a cyclopentyl group, a cyclohexyl group and the like.

The alkoxy group includes besides an aliphatic alkoxy group, an aryloxy group, an aralkyloxy group and the like. The aliphatic alkoxy group includes a linear or branched alkoxy group having 1 to 8 carbon atoms, preferably 1 to 4 carbon atoms. Specific examples thereof include a methoxy group, an ethoxy group, a propoxy group, an isopropoxy group, a n-butoxy group, a sec-butoxy group, an isobutoxy group, a tert-butoxy group and the like. The aryloxy group includes an aryloxy group having 6 to 14 carbon atoms, preferably 6 to 10 carbon atoms. Specific examples thereof include a phenoxy group, a naphthoxy group and the like. The aralkyloxy group includes an aralkyloxy group having 7 to 15 carbon atoms, preferably 7 to 11 carbon atoms. Specific examples thereof include a benzyloxy group, a 1-phenethyloxy group and the like.

The carboxy group includes a carboxy group derived from a carboxylic acid and having, for example, 2 to 18 carbon atoms. Specific examples thereof include an acetoxy group, a propionyloxy group, an acryloyloxy group, a butyryloxy group, a pivaloyloxy group, a pentanoyloxy group, a hexanoyloxy group, a lauroyloxy group, a stearoyloxy group, a benzoyloxy group and the like.

The siloxy group includes siloxy groups each substituted with a hydrocarbon having 1 to 12 carbon atoms. Specific examples thereof include a trimethylsiloxy group, a triethylsiloxy group, a triisopropylsiloxy group, a triphenylsiloxy group, a dimethyl-tert-butylsiloxy group, a diethylphenylsiloxy group, a diphenyl-tert-butylsiloxy group and the like.

The amino group includes an unsubstituted amino group and an amino group, in which a hydrogen atom on a nitrogen atom is replaced by a substituent such as an amino protecting group. Specific examples of the amino protecting group include an alkyl group having 1 to 8 carbon atoms, aryl group having 6 to 14 carbon atoms, aralkyl group having 7 to 15 carbon atoms, acyl group having 1 to 8 carbon atoms, alkoxycarbonyl group having 2 to 9 carbon atoms, aryloxycarbonyl group having 7 to 15 carbon atoms, aralkyloxycarbonyl group having 8 to 16 carbon atoms and a sulfonyl group having 1 to 14 carbon atoms.

Specific examples of the amino group having an alkyl substituent, that is, an alkylamino group, include mono or dialkylamino groups such as an N-methylamino group, an N,N-dimethylamino group, an N,N-diethylamino group, an N,N-diisopropylamino group and an N-cyclohexylamino group.

Specific examples of the amino group having an aryl substituent, that is, an arylamino group, include mono or diarylamino groups such as an N-phenylamino group, an N,N-diphenylamino group, an N-naphthylamino group and an N-naphthyl-N-phenylamino group.

Specific examples of the amino group having an aralkyl substituent, that is, an aralkylamino group, include mono or diaralkylamino groups such as an N-benzylamino group and an N,N-dibenzylamino group.

Specific examples of the amino group having an acyl substituent, that is, an acylamino group, include a formylamino group, an acetylamino group, a propionylamino group, an acryloylamino group, a pivaloylamino group, a pentanoylamino group, a hexanoylamino group and a benzoylamino group.

Specific examples of the amino group having an alkoxycarbonyl substituent, that is, an alkoxycarbonylamino group, include a methoxycarbonylamino group, an ethoxycarbonylamino group, a n-propoxycarbonylamino group, a n-butoxycarbonylamino group, a tert-butoxycarbonylamino group, a pentyloxycarbonylamino group, a hexyloxycarbonylamino group and the like.

Specific examples of the amino group having an aryloxycarbonyl group, that is, an aryloxycarbonylamino group, include a phenoxycarbonylamino group, a naphthyloxycarbonylamino group and the like.

Specific examples of the amino group having an aralkyloxycarbonyl substituent, that is, an aralkyloxycarbonylamino group, include a benzyloxycarbonylamino group and the like.

Specific examples of the amino group having a sulfonyl substituent, that is, a sulfonylamino group, include a methanesulfonylamino group and a p-toluenesulfonylamino group and the like.

Furthermore, the amino groups may have different substituents such as amino protecting groups. Specific examples thereof include a methylphenylamino group, a cyclopentyl-p-tolylamino group, an ethylisopropylamino group, isobutylnaphthylamino group, a benzylcyclohexylamino group and the like.

Furthermore, the alkyl group, alkoxy group, carboxy group and siloxy group represented by Lh and the alkyl group having 1 to 8 carbon atoms, aryl group, aralkyl group, acyl group, alkoxycarbonyl group, aryloxycarbonyl group, aralkyloxycarbonyl group and the sulfonyl group serving as the amino protecting group may further have a substituent. Specific examples thereof include an alkyl group having 1 to 12 carbon atoms, an alkoxy group having 1 to 12 carbon atoms, an aryl group having 6 to 12 carbon atoms, a heterocyclic group having 2 to 12 carbon atoms, a silyl group, a siloxy group, a carboxy group, an amino group and an amide group having carbon atoms 1 to 12, as well as polymer chains such as a 6,6-nylon chain, a vinyl polymer chain and a styrene polymer chain.

Lh is not necessarily an optically active substance.

A process for preparing an aluminum complex of the present invention will be described.

The aluminum complex of the present invention is obtained by reacting an aluminum compound represented by the general formula (1) and a diol compound represented by the general formula (2-A) or the general formula (2-B). Furthermore, by reacting an additive therewith, the aluminum complex can be obtained.

The aluminum complex of the present invention is obtained by reacting an aluminum compound represented by the general formula (1), a diol compound represented by the general formula (2-A) or the general formula (2-B), and a diol compound represented by the general formula (3-A) or the general formula (3-B).

A process for preparing an aluminum complex will be described separately with respect to the cases represented by the formula (1′) where n=1 and where n=2.

In the formula (1′) where l=1, for example, in an inert organic solvent, e.g., a hydrocarbon (hexane, heptane, benzene, toluene, xylene, etc.), an ether (diethyl ether, diisopropyl ether, tetrahydrofuran, etc.) or a halogenated hydrocarbon (dichloromethane, dichloroethane, chlorobenzene, bromotoluene, etc.), an aluminum compound of the general formula (1) and a diol compound (0.8 to 1.3 fold by mole relative to the aluminum compound) represented by the general formula (2-A) or the general formula (2-B) are reacted at a temperature of about −30 to 60° C., preferably about −10 to 40° C., more preferably about 0 to 30° C. for about 0.25 to 30 hours, preferably about 0.5 to 2 hours. In this manner, an aluminum complex can be easily synthesized. Furthermore, if necessary, an additive (0.1 to 2 fold by mole relative to the aluminum compound) is added and reacted at a temperature of about −30 to 60° C., preferably about −10 to 40° C., more preferably about 0 to 30° C. for about 0.25 to 30 hours, preferably about 0.5 to 2 hours. In this manner, an aluminum complex can be easily synthesized.

As an aluminum compound of the general formula (1) and a diol compound represented by the general formula (2-A) or the general formula (2-B), a solution thereof diluted with an inert organic solvent as mentioned above may be used.

When an aluminum complex is prepared using an additive, the additive is added for a reaction after an aluminum compound of the general formula (1) and a diol compound represented by the general formula (2-A) or the general formula (2-B) are reacted. An additive cannot be reacted by adding it simultaneously with an aluminum compound of the general formula (1) and a diol compound represented by the general formula (2-A) or the general formula (2-B).

When an additive is added, the additive may be added directly to a reaction solution of an aluminum compound of the general formula (1) and a diol compound of the general formula (2-A) or the general formula (2-B) or the additive may be diluted with an inert organic solvent as mentioned above and then added to the reaction solution. Furthermore, the reaction solution of an aluminum compound of the general formula (1) and a diol compound of the general formula (2-A) or the general formula (2-B) may be added to the additive.

In the formula (1′) where l=2, for example, in an inert organic solvent, e.g., a hydrocarbon (hexane, heptane, benzene, toluene, xylene, etc.), an ether (diethyl ether, diisopropyl ether, tetrahydrofuran, etc.) or a halogenation hydrocarbon (dichloromethane, dichloroethane, chlorobenzene, bromotoluene, etc.), an aluminum compound of the general formula (1) and a diol compound (0.8 to 1.3 fold by mole relative to the aluminum compound) represented by the general formula (2-A) or the general formula (2-B) are reacted at a temperature of about −30 to 60° C., preferably about −10 to 40° C., more preferably about 0 to 30° C. for about 0.25 to 30 hours, preferably about 0.5 to 2 hours. Next, a diol compound represented by the general formula (3-A) or the general formula (3-B) (0.4 to 0.7 fold by mole relative to the aluminum compound) is added and reacted at a temperature of about −30 to 60° C., preferably about −10 to 40° C., more preferably about 0 to 30° C. for about 0.25 to 30 hours, preferably about 0.5 to 2 hours. In this manner, the aluminum complex can be easily synthesized.

As an aluminum compound of the general formula (1), and a diol compound represented by the general formula (2-A), the general formula (2-B), the general formula (3-A) or the general formula (3-B), a diluted solution thereof with an inert organic solvent as mentioned above may be used.

Furthermore, in adding a diol compound represented by the general formula (3-A) or the general formula (3-B), a diol compound represented by the general formula (3-A) or the general formula (3-B) may be directly added to a reaction solution of an aluminum compound of the general formula (1) and a diol compound of the general formula (2-A) or the general formula (2-B) or a diol compound represented by the general formula (3-A) or the general formula (3-B) may be diluted with an inert organic solvent as mentioned above and then added to the reaction solution. Alternatively, the reaction solution of an aluminum compound of the general formula (1) and a diol compound of the general formula (2-A) or the general formula (2-B) may be added to a diol compound represented by the general formula (3-A) or the general formula (3-B).

A diol compound of the general formula (2-A) or the general formula (2-B) and a diol compound represented by the general formula (3-A) or the general formula (3-B) cannot be added for reaction simultaneously to an aluminum compound of the general formula (1). A diol compound represented by the general formula (3-A) or the general formula (3-B) must be added after an aluminum compound of the general formula (1) is reacted with a diol compound of the general formula (2-A) or the general formula (2-B) and subjected to a reaction.

However, in the formula (1′) where l=2 and where a diol compound of the general formula (2-A) or the general formula (2-B) and a diol compound represented by the general formula (3-A) or the general formula (3-B) are the same optically active compound, it is not necessary that the diol compound of the general formula (2-A) or the general formula (2-B) and a diol compound represented by the general formula (3-A) or the general formula (3-B) are separately reacted with an aluminum compound of the general formula (1). The diol compound of the general formula (2-A) or the general formula (2-B) and the diol compound represented by the general formula (3-A) or the general formula (3-B) may be simultaneously reacted with the aluminum compound of the general formula (1).

To describe more specifically, in an inert organic solvent as mentioned above, an aluminum compound of the general formula (1) and a diol compound (in an amount of larger than 1.3 fold by mole and less than 2.0 fold by mole relative to the aluminum compound) represented by the general formula (2-A) or the general formula (2-B), (or a diol compound represented by the general formula (3-A) or the general formula (3-B) may be used) may be reacted at a temperature of about −30 to 60° C., preferably about −10 to 40° C., more preferably about 0 to 30° C. for about 0.25 to 30 hours, preferably about 0.5 to 2 hours.

Specific examples of the additive include 2,6-diphenylphenol (sometimes referred to as DPP), o-phenylphenol (sometimes referred to as 2-PP), phenol, cyclopropanol, trimethylsilanol, tert-butyldimethylsilanol, tert-butanol, p-hydroxybenzoic acid, benzoic acid, benzylamine and N-methylphenylamine.

The ring closing reaction of an optical isomer mixture of a compound having both a formyl group and a double bond capable of causing a carbonyl-ene ring closing reaction in the same molecule can be conducted by use of the aluminum complex of the present invention. The aluminum complex of the present invention can conduct a selective ring closing reaction of a specific substrate, thereby increasing the proportion of d-form or l-form of a compound produced by ring closure or increasing the proportion of d-form or l-form of the optical isomer mixture which is not reacted by ring closure.

The compound having both a formyl group and a double bond capable of causing a carbonyl-ene ring closing reaction in the same molecule includes a compound represented by the general formula (4).

The compound produced by ring closure includes a compound represented by the general formula (5).

A compound used in the selective ring closing reaction of the present invention and represented by the general formula (4), and a compound produced by ring closure and represented by the general formula (5) will now be described.

In the compounds represented by the general formula (4) and (5), the alkyl group that may have a substituent and is represented by R⁹, R¹⁰, R¹¹, R¹², R¹³, R¹⁴ and R¹⁵ includes a linear or branched alkyl group having 1 to 8 carbon atoms, preferably 1 to 4 carbon atoms. Specific examples thereof include a methyl group, an ethyl group, a propyl group, an isopropyl group, a n-butyl group, a sec-butyl group, an isobutyl group and a tert-butyl group. The substituents that these alkyl groups have include an alkoxy group having 1 to 6 carbon atoms such as a methoxy group and an ethoxy group; and an aryl group having 6 to 14 carbon atoms such as a phenyl group, a naphthyl group and a tolyl group.

Furthermore, the protecting group of a hydroxy group that may be protected by a protecting group and represented by R¹¹ includes an acyl group having 1 to 8 carbon atoms such as an acetyl group, a benzoyl group and a methoxycarbonyl group; an aralkyl group having 7 to 15 carbon atoms such as a benzyl group; and a substituted silyl group having 3 to 30 carbon atoms such as a trimethylsilyl group and a t-butyldimethylsilyl group; and the like.

An example of the compound represented by the general formula (4) includes citronellal, 2,6-dimethyl-5-heptanal, 2,6,10-trimethyl-5,9-undecadienal, 3,7-dimethyl-2-methylene-6-octenal, 3,7,11-trimethyl-6,10-dodecadienal and the like, preferably an optically active citronellal, and more preferably 1-citronellal.

An example of the compound represented by the general formula (5) includes isopulegol, 2-(2-propenyl)-5-methylcyclopentanol, 2-(6-methyl-2,5-heptadien-2-yl)-5-methylcyclopentanol, 2-(6-methyl-1,5-heptadien-2-yl)-5-methylcyclopentanol, 2-methylene-3-methyl-6-(2-propenyl)cyclohexanol, 2-(6-methyl-2,5-heptadien-2-yl)-5-methylcyclohexanol, 2-(6-methyl-1,5-heptadien-2-yl)-5-methylcyclohexanol and the like, preferably an optically active isopulegol, and more preferably l-isopulegol.

Next, the selective ring closing reaction will be described.

The selective ring closing reaction for increasing the proportion of an optical isomer according to the present invention will be described below with reference to production of isopulegol by a ring closing reaction of citronellal using an aluminum complex obtained by using an optically active diol compound derived from tartaric acid, namely, 2,2-dimethyl-α,α,α′,α′-tetraphenyl-1,3-dioxolane-4,5-dimethanol (hereinafter sometimes referred to as TADDOL) as a diol compound represented by the general formula (2-A) or the general formula (3-A).

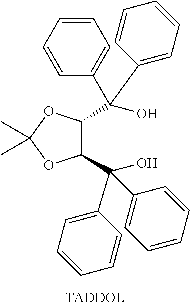

The present invention will be comprehensively described with reference to the examples below; however, the present invention is not limited to the substrate and product below.

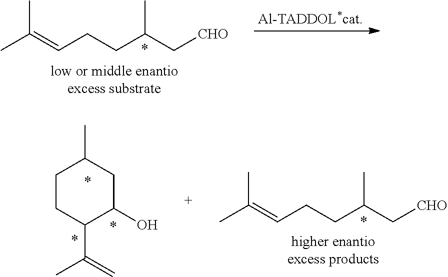

Al-TADDOL* cat. in the above represents an aluminum complex using an optically active TADDOL.

More specifically, citronellal having a low to middle optical purity is subjected to an enantio selective ring closing reaction using the aluminum-optically active diol complex of the present invention as a catalyst to produce isopulegol and citronellal having a higher optical purity than that of citronellal serving as the substrate.

The amount of aluminum catalyst used in the ring closing reaction of the present invention is about 0.05 to 10% by mole in terms of the atomic weight of aluminum (1 mole) relative to a compound represented by the general formula (4), for example, citronellal, preferably about 0.5 to 5% by mole, and further preferably about 0.7 to 2% by mole.

A process for preparing of the aluminum catalyst to be used in the ring closing reaction of the present invention is, for example, as follows:

(A) (a) an aluminum compound of the general formula (1) and a diol compound represented by the general formula (2-A) or the general formula (2-B) (0.8 to 1.3 fold by mole relative to the aluminum compound) are previously mixed in a reaction system and reacted, if necessary, an additive (0.1 to 2 fold by mole relative to the aluminum compound) is further added and reacted to prepare a catalyst, and thereafter citronellal is added (in-situ process);

(b) an aluminum compound of the general formula (1) and a diol compound represented by the general formula (2-A) or the general formula (2-B) (0.8 to 1.3 fold by mole relative to the aluminum compound) are previously mixed in a reaction system and reacted, and further a diol compound represented by the general formula (3-A) or the general formula (3-B) (0.4 to 0.7 fold by mole relative to the aluminum compound) is mixed and reacted to prepare a catalyst, and thereafter, citronellal is added (in-situ process); or

(B) a process of adding the catalyst prepared as mentioned above and citronellal separately at the time of a ring closing reaction.

The same results can be obtained by either process.

The temperature of the ring closing reaction is about −30 to 50° C., preferably about −10 to 30° C., and more preferably about 0 to 20° C. A compound represented by the general formula (5), for example, isopulegol, can be smoothly produced by conducting a reaction, while keeping the above temperature, for about 0.25 to 30 hours and preferably about 0.5 to 20 hours.

The ring closing reaction of the present invention can be conducted in the absence of a solvent or in the presence of an inert solvent.

The solvent to be used is not particularly limited as long as it does not significantly inhibit the reaction. Examples of the solvent include an aliphatic hydrocarbon organic solvent such as hexane, heptane and octane; an alicyclic hydrocarbon organic solvent such as cyclohexane and methylcyclohexane; an aromatic hydrocarbon organic solvent such as benzene, toluene and xylene; a halogenated hydrocarbon organic solvent such as dichloromethane, dichloroethane, chlorobenzene and bromotoluene; and an ether organic solvent such as diethyl ether, diisopropyl ether, dimethoxy ethane, tetrahydrofuran, dioxane and dioxolane; and the like. Of these, an organic solvent such as toluene and heptane is more preferably used.

Furthermore, an acid compound and a basic compound may be added at the time of the reaction. Specific examples of the acid compound include hydrochloric acid, sulfuric acid, acetic acid, citronellic acid, geranylic acid, nellic acid, acetic anhydride, propionic anhydride, maleic anhydride, succinic anhydride, pivaloyl acid anhydride and the like. Specific examples of the basic compound include sodium hydroxide, potassium carbonate, triethylamine and the like.

The use amount of these solvents is about 0 to 20 fold relative to the mass of citronellal and preferably 0.5 to 7 fold.

The ring closing reaction is preferably conducted in an inert gas atmosphere such as nitrogen gas or argon gas in order to smoothly conduct the ring closing reaction.

After completion of the ring closing reaction, conventional post treatments such as distillation, crystallization, and various types of chromatographic methods, are performed singly or in combination. In this manner, a reaction product can be purified. For example, to purify isopulegol, a distillation treatment is simply performed without performing cryogenic recrystallization. In this manner, highly purified isopulegol can be obtained. Furthermore, if the residue obtained after the distillation treatment is subjected to a general treatment with acid or alkali to remove aluminum impurities, etc. and then subjected to crystallization, a ligand can be used again.

### EXAMPLES

The present invention will be described in detail below with reference to the following non-limiting Examples.

Measurement of reaction products was performed by gas chromatography (GC) in the conditions as described below.

Analysis apparatus used: GC-2010 gas chromatography manufactured by Shimadzu Corporation

Column: conversion rate measurement, DB-WAX (0.25 mm×30 m) manufactured by Agilent,

Optical purity, beta-DEX-225 (0.25 mm×30 m) manufactured by

Supelco,

Detector: FID

Note that the optical purities of the citronellal isomers used in the present invention are as follows:

d-citronellal: 97.8% e.e.

l-citronellal: 96.6% e.e.

racemic citronellal: 0.74% e.e. 40%e.e. d-citronellal: 39.8% e.e. 60%e.e. d-citronellal: 59.6% e.e.

### Example 1

### Preparation of Aluminum Catalyst

In a nitrogen atmosphere, 181 mg (0.39 mmol) of (R,R)-2,2-dimethyl-α,α,α′,α′-tetraphenyl-1,3-dioxolane -4,5-dimethanol (hereinafter sometimes referred to as (R,R)-TADDOL) were placed in a 50 ml-Schlenk tube. After purged with nitrogen, methylene chloride (5 ml) and 0.4 ml of a triethylaluminum-exane solution (0.4 mmol, 1 mol/L) were sequentially added and stirred at room temperature for one hour. Thereafter, the solvent was distilled away to obtain a colorless to light orange solid substance. The solid substance was analyzed by ¹H-NMR. As a result, a peak of the aluminum complex was confirmed other than that of TADDOL.

¹H-NMR(DMSO-d₆): −0.49-1.20 (m, 16H), 4.79 (s, 2H), 7.10-7.47 (m, 16H), 7.55 (d, 5.4 Hz, 4H).

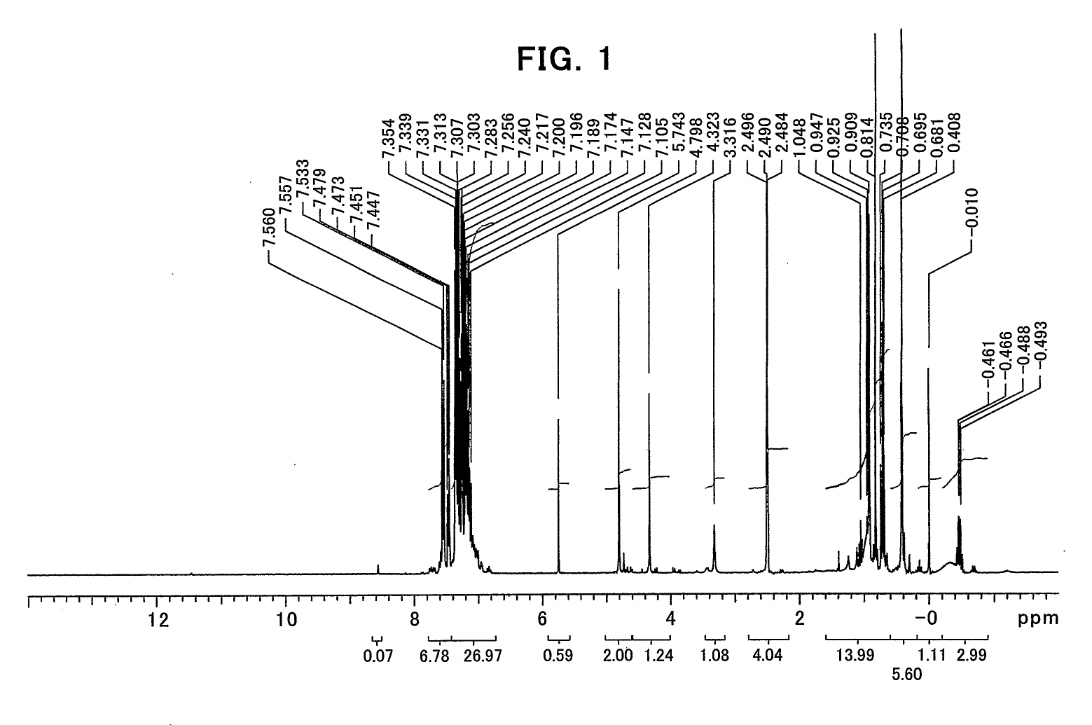

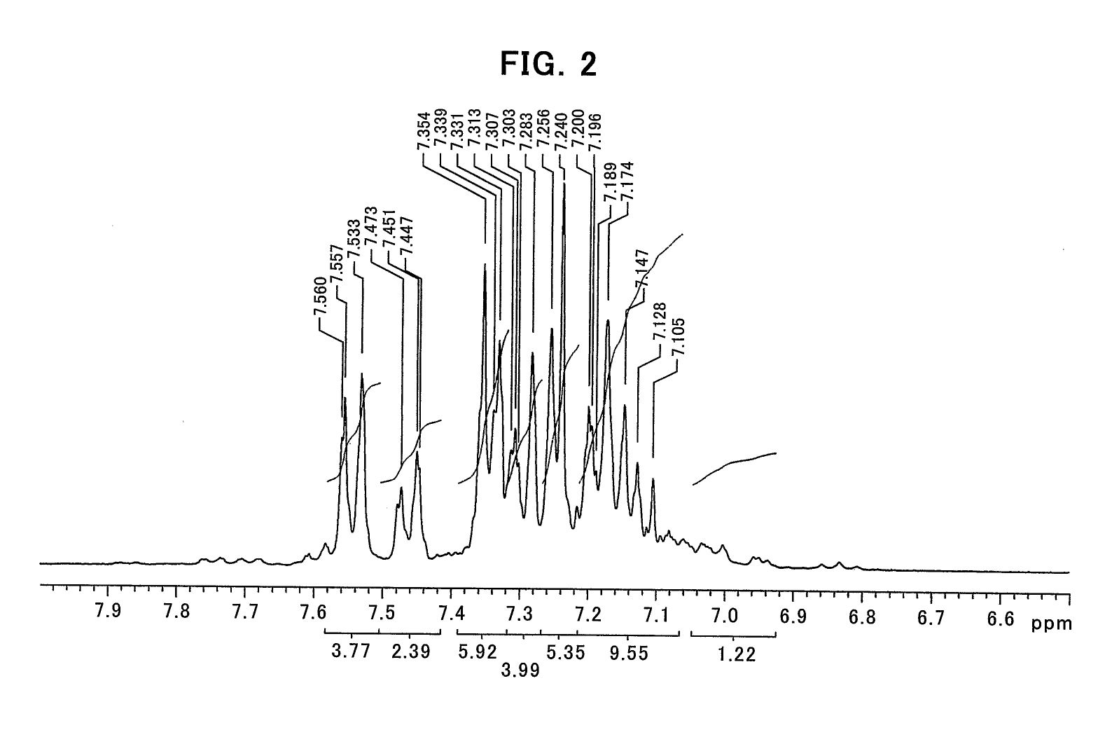

Furthermore, an NMR chart of the ligand and complex is shown in FIG. 1 and an enlarged view of a low magnetic field side thereof is shown in FIG. 2.

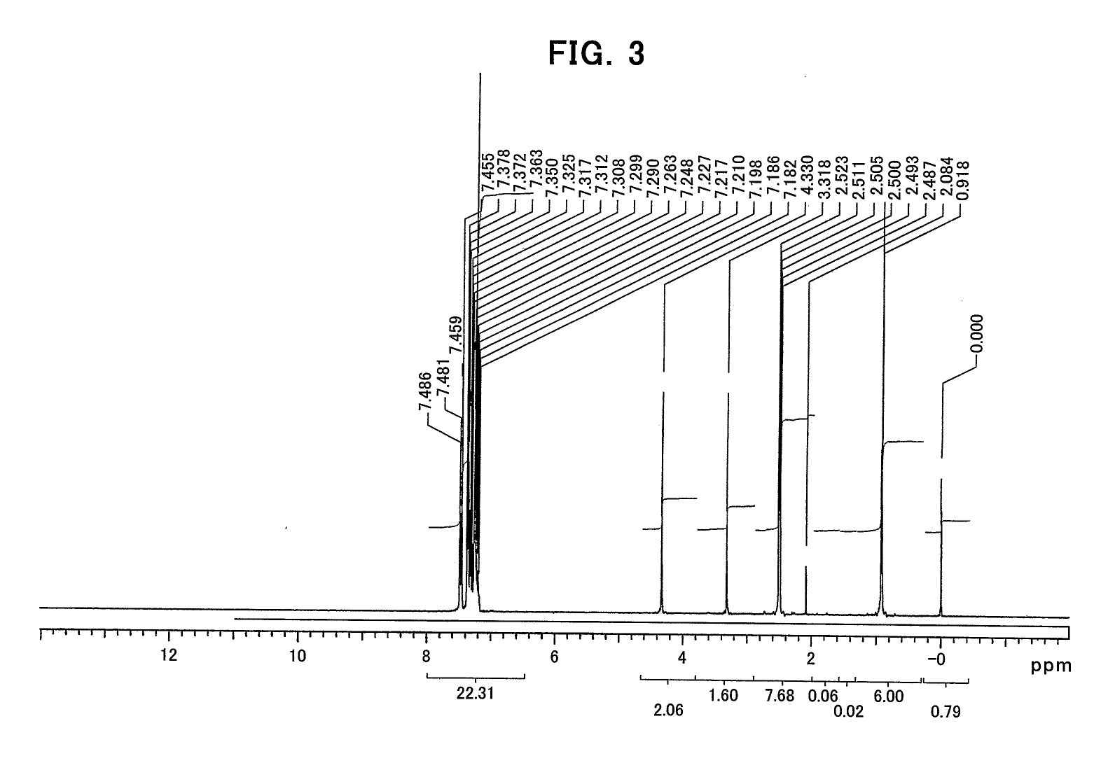

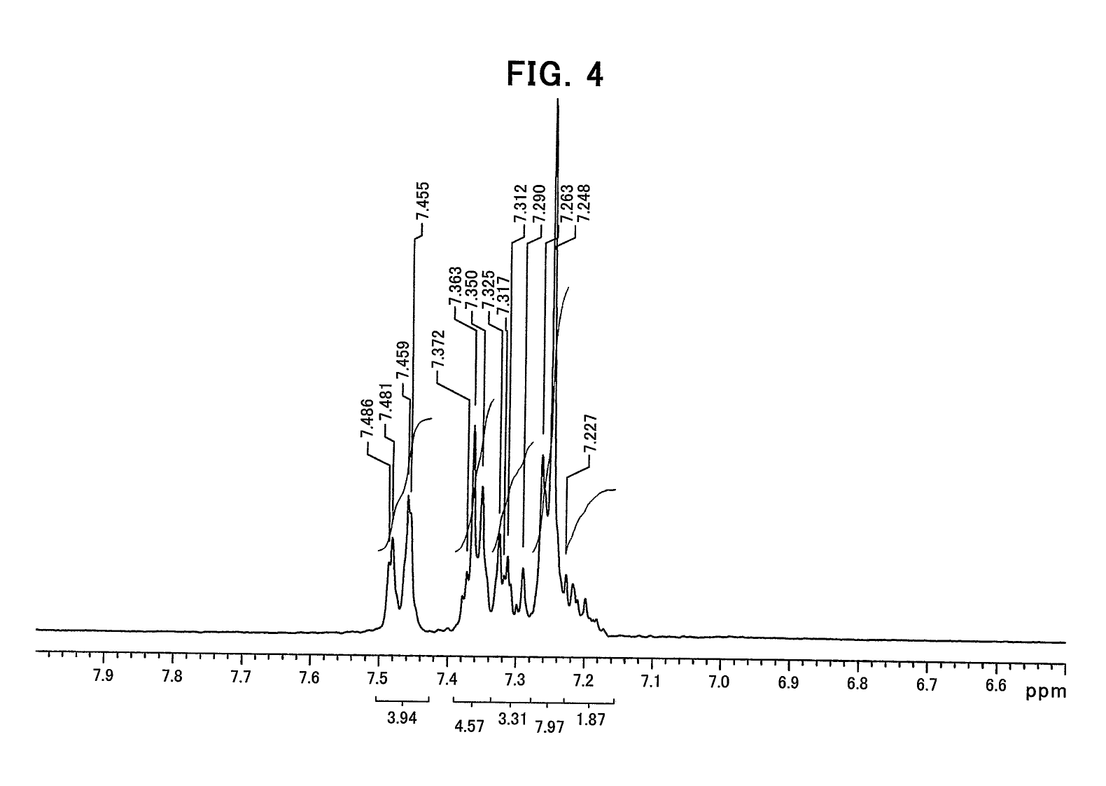

As a reference, an NMR chart of ligand (R,R)-TADDOL is shown in FIG. 3 and an enlarged view of a low magnetic field side thereof is shown in FIG. 4.

### Example 2

### Preparation of Aluminum Catalyst and Synthesis of d-isopulegol

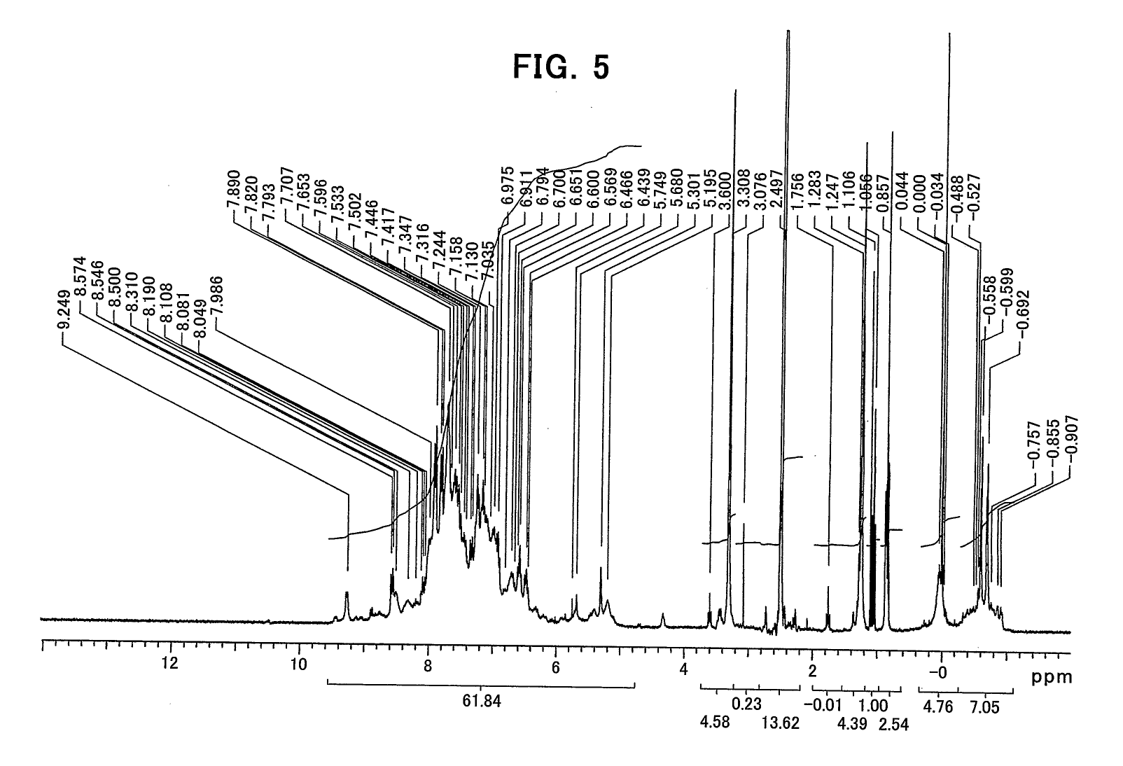

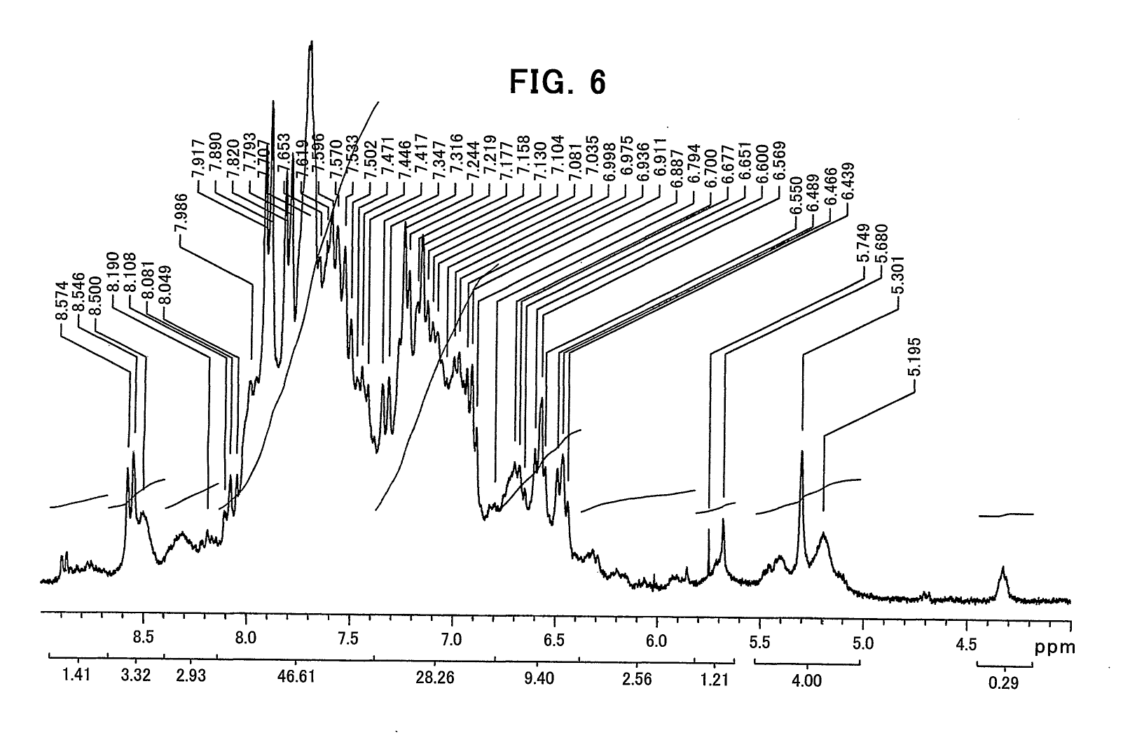

In a nitrogen atmosphere, 1.20 g (1.80 mmol) of (R,R)-2,2-dimethyl-α,α,α′,α′-(1-naphthyl)-1,3-dioxolane-4,5-dimethanol (hereinafter sometimes referred to as (R,R)-1-naphthyl TADDOL or (R,R)-1-NAPHTADDOL) were placed in a 100 ml-reaction flask. After purged with nitrogen, heptane (24 ml), 1.9 ml of a triethylaluminum.hexane solution (1.9 mmol, 1.0 mol/L) were sequentially added and stirred at room temperature for two hours to obtain a solid substance. The solid substance was filtrated under a nitrogen atmosphere, washed with a heptane solution, dried and thereafter analyzed by NMR. As a result, a peak of the aluminum complex was confirmed other than that of the ligand. An NMR chart of the ligand and complex is shown in FIG. 5 and an enlarged view of a low magnetic field side thereof is shown in FIG. 6.

The solid substance (700 mg) obtained above was added to l-citronellal (1.54 g, 10 mmol) cooled to 0 to 5° C. and stirred at 0 to 5° C. for one hour. After completion of the reaction, water (2 ml) and toluene (2 ml) were added and the organic layer was analyzed by gas chromatography. As a result, it was found that the substrate conversion rate was 90.2%, the isopulegol selectivity was 95.8% and the ratio of d-n-isopulegol to other isomers was 97.4:2.6.

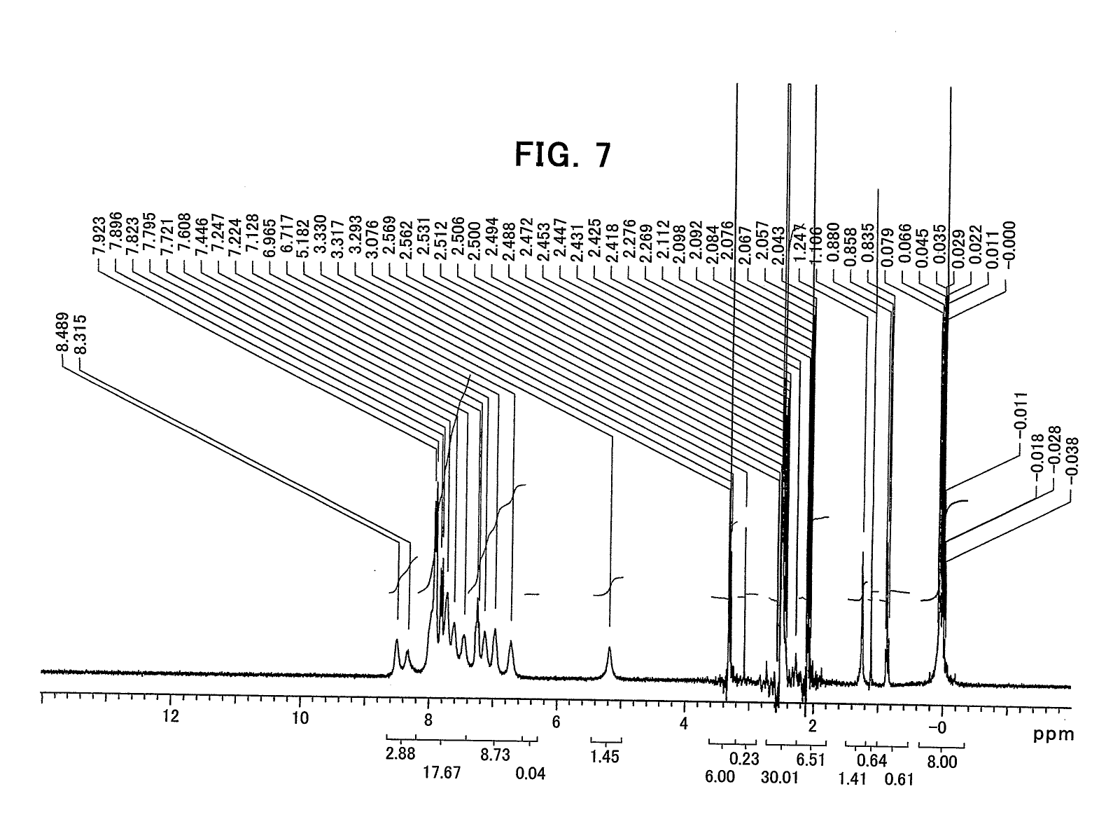

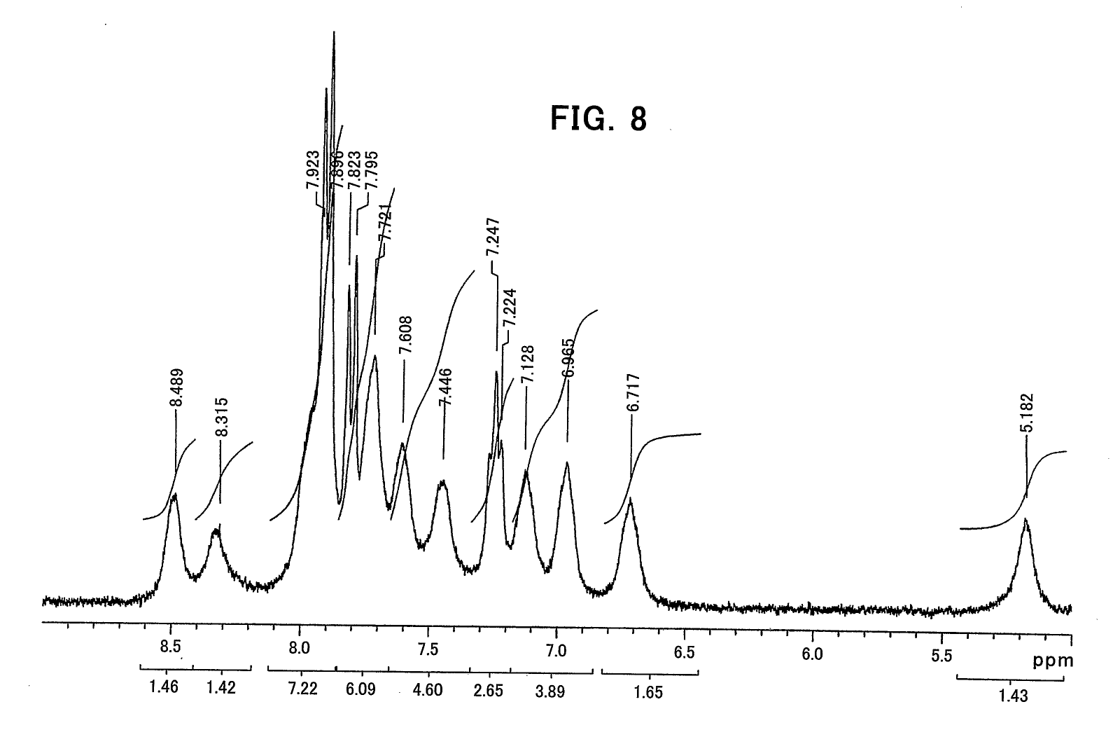

As a reference, an NMR chart of ligand (R,R)-1-NAPHTADDOL is shown in FIG. 7 and an enlarged view of a low magnetic field side thereof is shown in FIG. 8.

### Example 3

### Synthesis of l-isopulegol

216 mg (0.32 mmol) of (S,S)-2,2-dimethyl-α,α,α′,α′-(1-naphthyl)-1,3-dioxolane-4,5-dimethanol (hereinafter sometimes referred to as (S,S)-1-naphthyl TADDOL or (S,S)-1-NAPHTADDOL) were placed in a 50 ml-Schlenk tube. After purged with nitrogen, toluene (3 ml) and 0.32 ml of a triethylaluminum.toluene solution (0.32 mmol, 1.0 mol/L) were sequentially added and stirred at room temperature for one hour to obtain a catalyst solution. After the resultant catalyst solution was cooled to 0 to 5° C., d-citronellal (1.00 g, 6.5 mmol) was added dropwise and stirred at 0 to 5° C. for one hour. After completion of the reaction, water (2 ml) was added and the organic layer was analyzed by gas chromatography. As a result, it was found that the substrate conversion rate was 89.4%, the isopulegol selectivity was 87.4% and the ratio of l-n-isopulegol and other isomers was 96.1:3.9.

### Example 4

### Synthesis of l-isopulegol

216 mg (0.32 mmol) of (S,S)-1-naphthyl TADDOL were placed in a 50 ml-Schlenk tube. After purged with nitrogen, heptane (3 ml) and 0.32 ml of a triethylaluminum.toluene solution (0.32 mmol, 1.0 mol/L) were sequentially added and stirred at room temperature for one hour to obtain a catalyst solution. After the resultant catalyst solution was cooled to 0 to 5° C., d-citronellal (1.00 g, 6.5 mmol) was added dropwise and stirred at 0 to 5° C. for one hour. After completion of the reaction, water (2 ml) was added and the organic layer was analyzed by gas chromatography. As a result, it was found that the substrate conversion rate was 74.1%, the isopulegol selectivity was 85.0% and the ratio of l-n-isopulegol to other isomers was 93.2:6.8.

### Example 5

### Synthesis of d-isopulegol

389 mg (0.58 mmol) of (R,R)-1-naphthyl TADDOL were placed in a 50 ml-Schlenk tube. After purged with nitrogen, methylene chloride (3 ml) and 0.32 ml of a triethylaluminum.toluene solution (0.32 mmol, 1.0 mol/L) were sequentially added and stirred at room temperature for one hour to obtain a catalyst solution. After the resultant catalyst solution was cooled to 0 to 5° C., 1-citronellal (1.00 g, 6.5 mmol) was added dropwise and stirred at 0 to 5° C. for one hour. After completion of the reaction, water (2 ml) was added and the organic layer was analyzed by gas chromatography. As a result, it was found that the substrate conversion rate was 89.2%, the isopulegol selectivity was 90.2% and the ratio of d-n-isopulegol and other isomers was 92.9:7.1.

### Example 6

### Synthesis of d-isopulegol

389 mg (0.58 mmol) of (R,R)-1-naphthyl TADDOL were placed in a 50 ml-Schlenk tube. After purged with nitrogen, toluene (3 ml) and 0.32 ml of a triethylaluminum.toluene solution (0.32 mmol, 1.0 mol/L) were sequentially added and stirred at room temperature for one hour to obtain a catalyst solution. After the resultant catalyst solution was cooled to 0 to 5° C., l-citronellal (1.00 g, 6.5 mmol) was added dropwise and stirred at 0 to 5° C. for one hour. After completion of the reaction, water (2 ml) was added and the organic layer was analyzed by gas chromatography. As a result, it was found that the substrate conversion rate was 90.9%, the isopulegol selectivity was 88.7% and the ratio of d-n-isopulegol to other isomers was 97.4:2.6.

### Example 7

### Synthesis of l-isopulegol

389 mg (0.58 mmol) of (R,R)-1-naphthyl TADDOL were placed in a 50 ml-Schlenk tube. After purged with nitrogen, methylene chloride (3 ml) and 0.32 ml of a triethylaluminum.toluene solution (0.32 mmol, 1.0 mol/L) were sequentially added and stirred at room temperature for one hour to obtain a catalyst solution. After the resultant catalyst solution was cooled to 0 to 5° C., d-citronellal (1.00 g, 6.48 mmol) was added dropwise and stirred at 0 to 5° C. for one hour. After completion of the reaction, water (2 ml) was added and the organic layer was analyzed by gas chromatography. As a result, it was found that the substrate conversion rate was 54.8%, the isopulegol selectivity was 75.5% and the ratio of l-n-isopulegol to other isomers was 83.2:16.8.

### Example 8

### Synthesis of l-isopulegol from Racemic Citronellal

600 mg (0.9 mmol) of (S,S)-1-naphthyl TADDOL were placed in a 50 ml-Schlenk tube. After purged with nitrogen, toluene (11.6 ml) and 0.5 ml of a triethylaluminum.toluene solution (0.5 mmol, 1.0 mol/L) were sequentially added and stirred at room temperature for 2 hours to obtain a catalyst solution. After the catalyst solution was cooled to 0 to 5° C., a racemic citronellal (3.86 g, 25 mmol) was added dropwise and stirred at 0 to 5° C. for 7 hours. After completion of the reaction, water (2 ml) was added and the organic layer was analyzed by gas chromatography. As a result, it was found that the substrate conversion rate was 54.1%, the isopulegol selectivity was 95.0%, the enantio selectivity of l-citronellal was 23.6% e.e., and the enantio selectivity of 1-n-isopulegol was 22.3% e.e.

The aluminum complex of the present invention using (S,S)-1-naphthyl TADDOL (1.8-fold by mole relative to aluminum) as a ligand was excellent in selectivity of producing l-n-isopulegol by ring-closing d-citronellal of racemic citronellal.

### Example 9

### Synthesis of d-isopulegol from Racemic Citronellal

600 mg (0.9 mmol) of (R,R)-1-naphthyl TADDOL were placed in a 50 ml-Schlenk tube. After purged with nitrogen, toluene (11.6 ml) and 0.5 ml of a triethylaluminum.toluene solution (0.5 mmol, 1.0 mol/L) were sequentially added and stirred at room temperature for 2 hours to obtain a catalyst solution. After the catalyst solution was cooled to 0 to 5° C., racemic citronellal (3.86 g, 25 mmol) was added dropwise and stirred at 0 to 5° C. for 7 hours. After completion of the reaction, water (2 ml) was added and the organic layer was analyzed by gas chromatography. As a result, it was found that the substrate conversion rate was 58.9%, the isopulegol selectivity was 93.8%, the enantio selectivity of d-citronellal was 22.0% e.e. and the enantio selectivity of d-n-isopulegol was 21.1% e.e.

The aluminum complex of the present invention using (R,R)-1-naphthyl TADDOL (1.8-fold by mole relative to aluminum) as a ligand was excellent in selectivity of producing d-n-isopulegol by ring-closing l-citronellal of racemic citronellal.

### Example 10

### Synthesis of d-isopulegol from Racemic Citronellal

333 mg (0.5 mmol) of (R,R)-1-naphthyl TADDOL were placed in a 50 ml-Schlenk tube. After purged with nitrogen, toluene (11.6 ml) and 0.5 ml of a triethylaluminum.toluene solution (0.5 mmol, 1.0 mol/L) were sequentially added and stirred at room temperature for 2 hours to obtain a catalyst solution. After the catalyst solution was cooled to 0 to 5° C., racemic citronellal (3.86 g, 25 mmol) was added dropwise and stirred at 0 to 5° C. for 5 hours. After completion of the reaction, water (2 ml) was added and the organic layer was analyzed by gas chromatography. As a result, it was found that the substrate conversion rate was 46.7%, the isopulegol selectivity was 95.3%, the enantio selectivity of d-citronellal was 16.6% e.e. and the enantio selectivity of d-n-isopulegol was 19.8% e.e.

The aluminum complex of the present invention using (R,R)-1-naphthyl TADDOL (1.0-fold by mole relative to aluminum) as a ligand was excellent in selectivity of producing d-n-isopulegol by ring-closing l-citronellal of racemic citronellal.

### Example 11

### Synthesis of d-isopulegol from Racemic Citronellal

216 mg (0.32 mmol) of (R,R)-1-naphthyl TADDOL were placed in a 50 ml-Schlenk tube. After purged with nitrogen, methylene chloride (7.5 ml) and 0.32 ml of a triethylaluminum.toluene solution (0.32 mmol, 1.0 mol/L) were sequentially added and stirred at room temperature for 2 hours. To the reaction system, further 95.8 mg (0.38 mmol) of 2,6-diphenylphenol were added and stirred at room temperature for 2 hours to obtain a catalyst solution. After the catalyst solution was cooled to 0 to 5° C., racemic citronellal (2.5 g, 16.2 mmol) was added dropwise and stirred at 0 to 5° C. for 7 hours. After completion of the reaction, water (2 ml) was added and the organic layer was analyzed by gas chromatography. As a result, it was found that the substrate conversion rate was 39.1%, the isopulegol selectivity was 92.2%, the enantio selectivity of d-citronellal was 11.7% e.e. and the enantio selectivity of d-n-isopulegol was 32.7% e.e.

The aluminum complex of the present invention using (R,R)-1-naphthyl TADDOL (1.0-fold by mole relative to aluminum) and 2,6-diphenol (1.2-fold relative to the aluminum complex) which is an additive as a ligand was excellent in selectivity of producing d-n-isopulegol by ring-closing l-citronellal of racemic citronellal.

### Example 12

### Synthesis of 1-isopulegol from d-citronellal (40% e.e.)

600 mg (0.9 mmol) of (S,S)-1-naphthyl TADDOL were placed in a 50 ml-Schlenk tube. After purged with nitrogen, toluene (11.6 ml) and 0.5 ml of a triethylaluminum.toluene solution (0.5 mmol, 1.0 mol/L) were sequentially added and stirred at room temperature for 2 hours to obtain a catalyst solution. After the catalyst solution was cooled to 0 to 5° C., 40% e.e. of d-citronellal (3.86 g, 25 mmol) was added dropwise and stirred at 0 to 5° C. for 7 hours. After completion of the reaction, water (2 ml) was added and the organic layer was analyzed by gas chromatography. As a result, it was found that the substrate conversion rate was 67.2%, the isopulegol selectivity was 96.9%, the enantio selectivity of l-citronellal was 12.7% e.e. and the enantio selectivity of l-n-isopulegol was 55.4% e.e. The ratio of l-n-isopulegol to other isomers was 98.9:1.1.

### Example 13

### Synthesis of l-isopulegol from d-citronellal (60% e.e.)

600 mg (0.9 mmol) of (S,S)-1-naphthyl TADDOL were placed in a 50 ml-Schlenk tube. After purged with nitrogen, toluene (11.6 ml) and 0.5 ml of a triethylaluminum.toluene solution (0.5 mmol, 1.0 mol/L) were sequentially added at room temperature and stirred for 2 hours to obtain a catalyst solution. After the catalyst solution was cooled to 0 to 5° C., 60% e.e. of d-citronellal (3.86 g, 25 mmol) was added dropwise and stirred at 0 to 5° C. for 7 hours. After completion of the reaction, water (2 ml) was added and the organic layer was analyzed by gas chromatography. As a result, it was found that the substrate conversion rate was 76.7%, the isopulegol selectivity was 94.8%, the enantio selectivity of l-citronellal was 29.0% e.e. and the enantio selectivity of l-n-isopulegol was 69.8% e.e. The ratio of l-n-isopulegol to other isomers was 98.1:1.9.

### Example 14 to 16

### Synthesis of Isopulegol with Aluminum Catalyst

A predetermined amount of diol compound (represented by L¹H₂ in Table 1) of the general formula (2-A) was placed in a 50 ml-Schlenk tub. After purged with nitrogen, a solvent (3 ml) and triethylaluminum (0.32 mmol) were sequentially added and stirred at room temperature for one hour. Further, an additive was added in a predetermined amount and stirred at room temperature for one hour to obtain a catalyst solution. After the catalyst solution was cooled to 0 to 5° C., citronellal (1.00 g, 6.48 mmol) was added dropwise and stirred at 0 to 5° C. for one hour. After completion of the reaction, water (2 ml) was added and the organic layer was analyzed by gas chromatography.

The results are shown in Table 1.

In the table, conv. represents the conversion rate of citronellal; sel. represents the selectivity to isopulegol; and n-sel. represents the selectivity to n-isopulegol.

In the table, Tol represents toluene.

In the table, (S,S)-1-NAPHTADDOL, DPP, 2-PP (also referred to as 2-Phenylphenol) represent the following compounds, respectively.

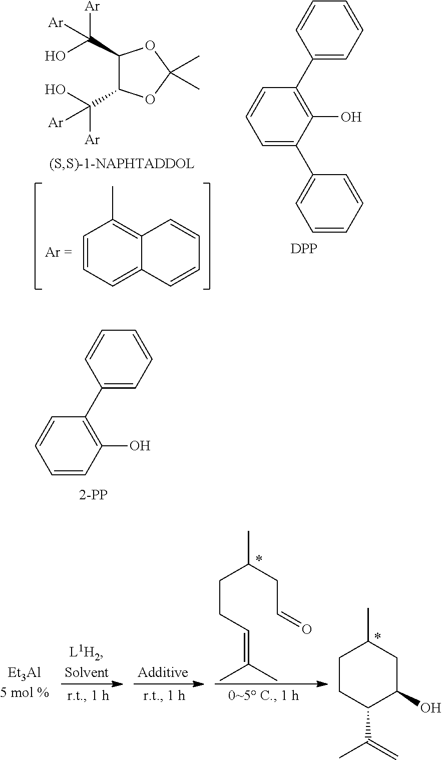

The above Additive represents an additive.

h represents hour.

TABLE 1

| | | | | Additive | | | | |
|---|---|---|---|---|---|---|---|---|
| | | | | (fold by | | | | |
| | | | L¹H₂ | mole | | | | |
| | | | (fold by mole | relative to | Conv. | Sel. | n-sel. | n-isopulegol |
| Example | Solvent | Citronellal | relative to Et₃Al) | Et₃Al) | (%) | (%) | (%) | produced |
| 14 | CH₂Cl₂ | d | (S,S)-1-NAPHTADDOL | DPP | 98.6 | 97.7 | 96.6 | l |
| | | | (1) | (0.8) | | | | |
| 15 | CH₂Cl₂ | l | (S,S)-1-NAPHTADDOL | DPP | 67.0 | 90.8 | 93.7 | d |
| | | | (1) | (0.8) | | | | |
| 16 | Tol | d | (S,S)-1-NAPHTADDOL | 2-PP | 96.8 | 75.4 | 90.3 | l |
| | | | (1) | (0.8) | | | | |

1-43. (canceled)

14. An aluminum complex represented by the general formula (1′) below:
[Alₗ(L¹)ₗ(L²)ₘ(Lh)ₙ]ₖ (1′)
wherein in the formula (1′), l represents an integer of 1 or 2, with the proviso that when l=1, m=0 and n=1, and when l=2, m=1 and n=0; k represents a natural number; L¹ represents a ligand represented by the formula (2-A′) below or the formula (2-B′) below; L² represents a ligand represented by the formula (3-A′) or (3-B′) below; Lh represents an alkyl group, an alkoxy group, a carboxy group, a siloxy group, an amino group, a fluorine atom, a bromine atom or an iodine atom, 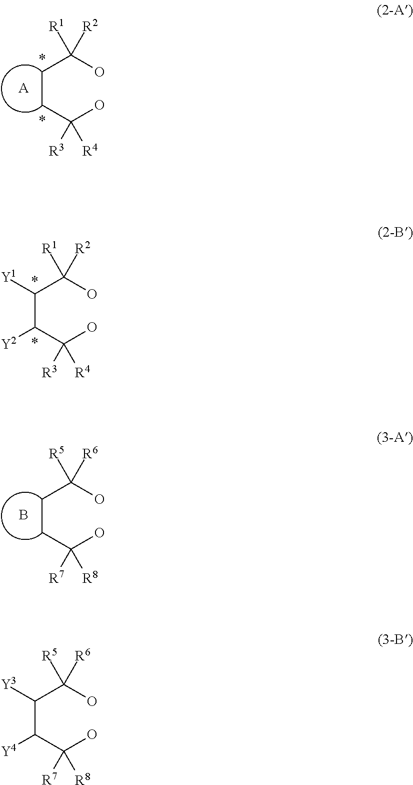
in the formula (2-A′), R¹, R², R³ and R⁴ each independently represent an aryl group that may have a substituent, a heterocyclic group that may have a substituent, an aliphatic chain that may have a substituent or an alicyclic group that may have a substituent, and R¹ and R², and R³ and R⁴ may be taken together to form a ring, and ring A is a 3- to 8-membered ring that may have a hetero element and symbol * represents an optically active asymmetric carbon atom,
in the formula (2-B′), R¹, R², R³ and R⁴ each independently represent an aryl group that may have a substituent, a heterocyclic group that may have a substituent, an aliphatic chain that may have a substituent or an alicyclic group that may have a substituent. R¹ and R², and R³ and R⁴ may be taken together to form a ring. Y¹ and Y² each independently represent an aliphatic chain that may have a substituent, an alicyclic group that may have a substituent, an aryl group that may have a substituent, a heterocyclic group that may have a substituent, an alkoxy group, a siloxy group that may have a substituent or a carboxy group, and symbol * represents an optically active asymmetric carbon atom,
in the formula (3-A′), R⁵, R⁶, R⁷, and R⁸ each independently represent an aryl group that may have a substituent, a heterocyclic group that may have a substituent, an aliphatic chain that may have a substituent or an alicyclic group that may have a substituent, and R⁵ and R⁶, and R⁷ and R⁸ may be taken together to form a ring, and ring B is a 3- to 8-membered ring that may have a hetero element, and
in the formula (3-B′), R⁵, R⁶, R⁷ and R⁸ each independently represent an aryl group that may have a substituent, a heterocyclic group that may have a substituent, an aliphatic chain that may have a substituent, or an alicyclic group that may have a substituent. R⁵ and R⁶, and R⁷ and R⁸ may be taken together to form a ring. Y³ and Y⁴ each independently represent an aliphatic chain that may have a substituent, an alicyclic group that may have a substituent, an aryl group that may have a substituent, a heterocyclic group that may have a substituent, an alkoxy group, a siloxy group that may have a substituent, or a carboxy group.

15. A process for producing the aluminum complex according to claim 14, said process comprising the step of reacting an aluminum compound represented by the general formula (1) below:
Al(Lg)₃ (1)
wherein in the formula (1), Lg represents an alkyl group, an alkoxy group or a halogen atom,
with a diol compound represented by the general formula (2-A) below or the general formula (2-B) below: 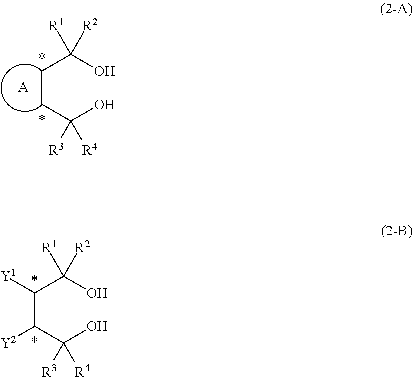
wherein in the formula (2-A), R¹, R², R³, R⁴, ring A and symbol * have the same meanings as defined in the formula (2-A′) of claim 1, and in the formula (2-B), R¹, R², R³, R⁴, Y¹, Y² and symbol * have the same meanings as defined in the formula (2-B′) of claim 1.

16. The process for producing the aluminum complex according to claim 15, wherein in the reaction an additive is added.

17. A process for producing the aluminum complex according to claim 14, said process comprising the step of reacting an aluminum compound represented by the general formula (1) below:
Al(Lg)₃ (1)
wherein in the formula (1), Lg represents an alkyl group, an alkoxy group or a halogen atom,
with a diol compound represented by the general formula (2-A) below or the general formula (2-B) below, and a diol compound represented by the general formula (3-A) below or the general formula (3-B) below: 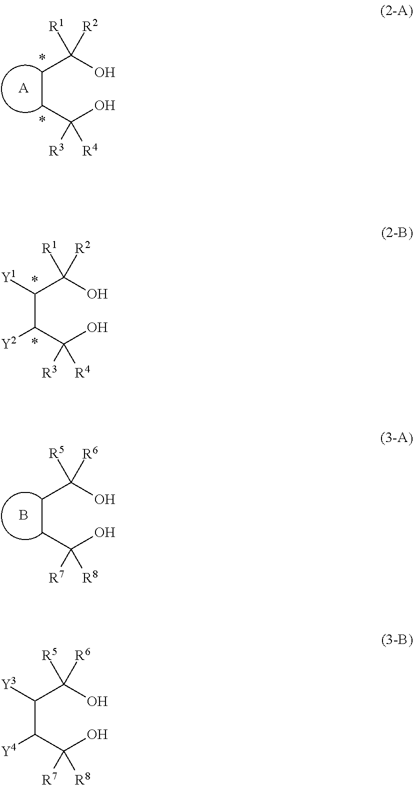
wherein in the formula (2-A), R¹, R², R³, R⁴ ring A and symbol * have the same meanings as defined in the formula (2-A′) of claim 1,
in the formula (2-B), R¹, R², R³, R⁴, Y¹, Y² and symbol * have the same meanings as defined in the formula (2-B′) of claim 1,
in the formula (3-A), R⁵, R⁶, R⁷, R⁸ and ring B have the same meanings as defined in the formula (3-A′) of claim 1, and
in the formula (3-B), R⁵, R⁶, R⁷, R⁸, Y³ and Y⁴ have the same meanings as defined in the formula (3-B′) of claim 1.

18. The process for producing the aluminum complex according to claim 15, wherein the diol compound represented by the general formula (2-A) or the general formula (2-B) is an optically active substance derived from tartaric acid.

19. The process for producing the aluminum complex according to claim 17, wherein the diol compound represented by the general formula (3-A) or the general formula (3-B) is an optically active substance derived from tartaric acid.

20. A process for producing an optically active compound, said process comprising the step of subjecting an optical isomer mixture of a compound having both a formyl group and a double bond capable of causing a carbonylene ring closing reaction in the same molecule to a ring closing reaction in the presence of the aluminum complex according to claim 14,
wherein the optically active compound is enriched with either a d-form or l-form compound produced by the ring closing reaction of the compound having both the formyl group and the double bond.

21. The production process according to claim 20, wherein the compound having both the formyl group and the double bond capable of causing the carbonyl-ene ring closing reaction in the same molecule is a compound represented by the general formula (4) below: 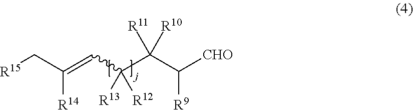
wherein in the formula (4), j represents an integer of 1 or 2; R⁹, R¹⁰and R¹² each independently represent a hydrogen atom or an alkyl group that may have a substituent; R¹¹ represents an alkyl group that may have a substituent or a hydroxy group that may be protected with a protecting group; R¹³, R¹⁴ and R¹⁵ each independently represent a hydrogen atom or an alkyl group that may have a substituent; and a wavy line represents an E or Z conformation.

22. The production process according to claim 20, wherein the compound produced by ring closure is a compound represented by the general formula (5) below: 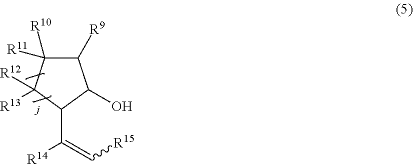
wherein in the formula (5), j represents an integer of 1 or 2; R⁹, R¹⁰ and R¹² each independently represent a hydrogen atom or an alkyl group that may have a substituent; R¹¹ represents an alkyl group that may have a substituent or a hydroxy group that may be protected with a protecting group; R¹³, R¹⁴ and R¹⁵ each independently represent a hydrogen atom or an alkyl group that may have a substituent; and a wavy line represents an E or Z conformation.

23. The production process according to claim 20, wherein the compound having both the formyl group and the double bond capable of causing the carbonyl-ene ring closing reaction in the same molecule is optically active citronellal and the compound produced by ring closure is optically active isopulegol.

24. The production process according to claim 23, wherein the optically active isopulegol is l-isopulegol.

25. The production process according to claim 23, wherein the optically active citronellal is l-citronellal.

26. A process for enriching either d-form or l-form in an optical isomer mixture of a compound having both a formyl group and a double bond capable of causing a carbonyl-ene ring closing reaction in the same molecule, said process comprising the step of subjecting the optical isomer mixture to a ring closing reaction in the presence of the aluminum complex according to claim 14,
wherein either d-form or l-form is not reacted by ring closure.
